SEQUENCE LISTING (8 sequences)

SEQ ID NO: 1 - protein, 1270 aa, Homo sapien
MGDVKNFLYAWCGKRKMTPSYEIRAVGNKNRQKFMCEVQVEGYNYTGMGNSTNKKDAQSNAARDFVNYLVRINEIKSEEVPAFGVASPPPLTDTPDTTANAEGDLPTTMGGPLPPHLALKAENNSEVGASGYGVPGPTWDRGANLKDYYSRKEEQEVQATLESEEVDLNAGLHGNWTLENAKARLNQYFQKEKIQGEYKYTQVGPDHNRSFIAEMTIYIKQLGRRIFAREHGSNKKLAAQSCALSLVRQLYHLGVVEAYSGLTKKKEGETVEPYKVNLSQDLEHQLQNIIQELNLEILPPPEDPSVPVALNIGKLAQFEPSQRQNQVGVVPWSPPQSNWNPWTSSNIDEGPLAFATPEQISMDLKNELMYQLEQDHDLQAILQERELLPVKKFESEILEAISQNSVVIIRGATGCGKTTQVPQFILDDFIQNDRAAECNIVVTQPRRISAVSVAERVAFERGEEPGKSCGYSVRFESILPRPHASIMFCTVGVLLRKLEAGIRGISHVIVDEIHERDINTDFLLVVLRDVVQAYPEVRIVLMSATIDTSMFCEYFFNCPIIEVYGRTYPVQEYFLEDCIQMTHFVPPPKDKKKKDKDDDGGEDDDANCNLICGDEYGPETRLSMSQLNEKETPFELIEALLKYIETLNVPGAVLVFLPGWNLIYTMQKHLEMNPHFGSHRYQILPLHSQIPREEQRKVFDPVPVGVTKVILSTNIAETSITINDVVYVIDSCKQKVKLFTAHNNMTNYSTVWASKTNLEQRKGRAGRSTAGFCFHLCSRARFERLETHMTPEMFRTPLHEIALSIKLLRLGGIGQFLAKAIEPPPLDAVIEAEHTLRELDALDANDELTPLGRILAKLPIEPRFGKMMIMGCIFYVGDAICTIAAATCFPEPFINEGKRLGYIHRNFAGNRFSDHVALLSVFQAWDDARMGGEEAEIRFCEHKRLNMATLRMTWEAKVQLKEILINSGFPEDCLLTQVFTNTGPDNNLDVVISLLAFGVYPNVCYHKEKRKILTTEGRNALIHKSSVNCPFSSQDMKYPSPFFVFGEKIRTRAISAKGMTLVPPLQLLLFASKKVQSDGQIVLVDDWIKLQISHEAAACITGLRAAMEALVVEVTKQPAIISQLDPVNERMLNMIRQISRPSAAGINLMIGSTRYGDGPRPPKMARYDNGSGYRRGGSSYSGGGYGGGYSSGGYGSGGYGGSANSFRAGYGAGVGGGYRGVSRGGFRGNSGGDYRGPSGGYRGSGGFQRGGGRGAYGTGYFGQGRGGGGY

SEQ ID NO: 2 - DNA, 3810 bp, Home sapien
atgggtgacgttaaaaattttctgtatgcctggtgtggcaaaaggaagatgaccccatcctatgaaattagagcagtggggaacaaaaacaggcagaaattcatgtgtgaggttcaggtggaaggttataattacactggcatgggaaattccaccaataaaaaagatgcacaaagcaatgctgccagagactttgttaactatttggttcgaataaatgaaataaagagtgaagaagttccagcttttggggtagcatctccgcccccacttactgatactcctgacactacagcaaatgctgaaggagatttaccaacaaccatgggaggacctcttcctccacatctggctctcaaagcagaaaataattctgaggtaggggcctctggctatggtgttcctgggcccacctgggaccgaggagccaacttgaaggattactactcaagaaaggaagaacaagaagtgcaagcgactctagaatcagaagaagtggatttaaatgctgggcttcatggaaactggaccttggaaaatgctaaagctcgtctaaaccaatattttcagaaagaaaagatccaaggagaatataagtacacccaagtgggtcctgatcacaacaggagctttattgcagaaatgaccatttatatcaagcagctgggcagaaggatttttgcacgagaacatggatcaaataagaaattggcagcacagtcctgtgccctgtcacttgtcagacaactgtaccatcttggagtggttgaagcttactccggacttacaaagaagaaggaaggagagacagtggagccttacaaagtaaacctctctcaagatttagagcatcagctgcaaaacatcattcaagagctaaatcttgagattttgcccccgcctgaagatccttctgtgccagttgcactcaacattggcaaattggctcagttcgaaccatctcagcgacaaaaccaagtgggtgtggttccttggtcacctccacaatccaactggaatccttggactagtagcaacattgatgaggggcctctggcttttgctactccagagcaaataagcatggacctcaagaatgaattgatgtaccagttggaacaggatcatgatttgcaagcaatcttgcaggagagagagttactgcctgtgaagaaatttgaaagtgagattctggaagcaatcagccaaaattcagttgtcattattagaggggctactggatgtgggaaaaccacacaggttccccagttcattctagatgactttatccagaatgaccgagcagcagagtgtaacatcgtagtaactcagcccagaagaatcagtgcggtttctgtggcagagcgagttgcatttgaaagaggagaagagcctggaaaaagctgtggctacagcgttcgatttgagtctatacttcctcgtcctcatgccagtataatgttttgtactgtaggtgtgctcctgagaaaattagaagcaggcattcgaggaatcagtcatgtaattgtagatgaaatacatgaaagagatattaatactgacttccttctggtagtactgcgtgatgttgttcaggcttatcctgaagttcgcattgttcttatgtctgctactattgataccagcatgttttgtgaatatttcttcaattgccccatcattgaagtttatgggaggacttacccagttcaagaatattttctggaagactgcattcagatgacccactttgttcctccaccaaaagacaaaaagaagaaggataaggatgatgatggtggtgaggatgatgatgcaaattgcaacttgatctgtggtgatgaatatggtccagaaacaaggttgagcatgtctcaattgaacgaaaaggaaactccttttgaactcatcgaggctctacttaagtacattgaaacccttaatgttcctggagctgtgttggtttttttgcctggctggaatctgatttatactatgcagaagcatttggaaatgaatccacattttggaagccatcggtatcagattctacccctgcattctcagattcctcgagaggaacagcgcaaagtgtttgatccagtaccagttggagtaaccaaggttattttgtccacaaatattgctgaaacaagcattaccataaacgatgttgtttatgtcattgactcctgcaagcagaaagtgaaactcttcactgctcacaacaatatgaccaactattctaccgtatgggcatcaaaaacaaaccttgagcaacggaaagggcgagctggccggagtacggctggattctgctttcacctgtgcagccgagctcgttttgagagacttgaaacccacatgacaccagagatgttccgaacaccattgcatgaaattgctcttagcataaaacttctgcgtctaggaggaattggccaatttctggccaaagcaattgaacctccccctttggatgctgtgattgaagcagaacacactcttagagagcttgatgcattagatgccaatgatgagttgactcctttgggacgaatcctggctaaactccccattgagcctcgttttggcaaaatgatgataatggggtgtattttctacgtgggagatgctatctgtaccattgctgctgctacctgctttccagagcctttcatcaatgaaggaaagcggctgggctatatccatcgaaattttgctggaaacagattttctgatcacgtagcccttttatcagtattccaagcctgggatgatgctagaatgggtggagaagaagcagagatacgtttttgtgagcacaaaagacttaatatggctacactaagaatgacctgggaagccaaagttcagctcaaagagattttgattaattctgggtttccagaagattgtttgttgacacaagtgtttactaacactggaccagataataatttggatgttgttatctccctcctggcctttggtgtgtaccccaatgtatgctatcataaggaaaagaggaagattctcaccactgaagggcgtaatgcacttatccacaaatcatctgttaattgtccttttagtagccaagacatgaagtacccatctcccttctttgtatttggtgaaaagattcgaactcgagccatctctgctaaaggcatgactttagtaccccccctgcagttgcttctctttgcctccaagaaagtccaatctgatgggcagattgtgcttgtagatgactggattaaactgcaaatatctcatgaagctgctgcctgtatcactggtctccgggcagccatggaggctttggttgttgaagtaaccaaacaacctgctatcatcagccagttggaccccgtaaatgaacgtatgctgaacatgatccgtcagatctctagaccctcagctgctggtatcaaccttatgattggcagtacacggtatggagatggtccacgtcctcccaagatggcccgatacgacaatggaagcggatatagaaggggaggttctagttacagtggtggaggctatggcggtggctatagcagtggaggctatggtagcggaggctatggtggcagcgccaactcctttcgggcaggatatggtgcaggtgttggtggaggctatagaggagtttcccgaggtggctttagaggcaactctggaggagactacagagggcctagtggaggctacagaggatctgggggattccagcgaggaggtggtaggggggcctatggaactggctactttggacagggaagaggaggtggcggctat

SEQ ID NO: 3 - protein, 894 aa, Homo sapien [sequence omitted]

SEQ ID NO: 4 - DNA, 2685 bp, Homo sapien
atgcgtccaatgcgaatttttgtgaatgatgaccgccatgtgatggcaaagcattcttccgtttatccaacacaagaggagctggaggcagtccagaacatggtgtcccacacggagcgggcgctcaaagctgtgtccgactggatagacgagcaggaaaagggtagcagcgagcaggcagagtccgataacatggatgtgcccccagaggacgacagtaaagaaggggctggggaacagaagacggagcacatgaccagaaccctgcggggagtgatgcgggtgggcctggtggcaaagtgcctcctactcaagggggacttggatctggagctggtgctgctgtgtaaggagaagcccacaaccgccctcctggacaaggtggccgacaacctggccatccagcttgctgctgtaacagaagacaagtacgaaatactgcaatctgtcgacgatgctgcgattgtgataaaaaacacaaaagagcctccattgtccctgaccatccacctgacatcccctgttgtcagagaagaaatggagaaagtattagctggagaaacgctatcagtcaacgaccccccggacgttctggacaggcagaaatgccttgctgccttggcgtccctccgacacgccaagtggttccaggccagagccaacgggctgaagtcttgtgtcattgtgatccgggtcttgagggacctgtgcactcgcgtgcccacctggggtcccctccgaggctggcctctcgagctcctgtgtgagaaatccattggcacggccaacagaccgatgggtgctggcgaggccctgcggagagtgctggagtgcctggcgtcgggcatcgtgatgccagatggttctggcatttatgacccttgtgaaaaagaagccactgatgctattgggcatctagacagacagcaacgggaagatatcacacagagtgcgcagcacgcactgcggctcgctgccttcggccagctccataaagtcctaggcatggaccctctgccttccaagatgcccaagaaaccaaagaatgaaaacccagtggactacaccgttcagatcccaccaagcaccacctatgccattacgcccatgaaacgcccaatggaggaggacggggaggagaagtcgcccagcaaaaagaagaagaagattcagaagaaagaggagaaggcagagcccccccaggctatgaatgccctgatgcggttgaaccagctgaagccagggctgcagtacaagctggtgtcccagactgggcccgtccatgcccccatctttaccatgtctgtggaggttgatggcaattcattcgaggcctctgggccctccaaaaagacggccaagctgcacgtggccgttaaggtgttacaggacatgggcttgccgacgggtgctgaaggcagggactcgagcaagggggaggactcggctgaggagaccgaggcgaagccagcagtggtggcccctgccccagtggtagaagctgtctccacccctagtgcggcctttccctcagatgccactgccgagcaggggccgatcctgacaaagcacggcaagaacccagtcatggagctgaacgagaagaggcgtgggctcaagtacgagctcatctccgagaccgggggcagccacgacaagcgcttcgtcatggaggtcgaagtggatggacagaagttccaaggtgctggttccaacaaaaaggtggcgaaggcctacgctgctcttgctgccctagaaaagcttttccctgacacccctctcgcccttgatgccaacaaaaagaagagagccccagtacccgtcagagggggaccgaaatttgctgctaagccacataaccctggcttcggcatgggaggccccatgcacaacgaagtgcccccaccccccaaccttcgagggcggggaagaggcgggagcatccggggacgagggcgcgggcgaggatttggtggcgccaaccatggaggctacatgaatgccggtgctgggtatggaagctatgggtacggaggcaactctgcgacagcaggctacagtcagttctacagcaacggagggcattctgggaatgccagtggcggtggcggcgggggcggtggtggctcctccggctatggctcctactaccaaggtgacaactacaactcaccggtgcccccaaaacacgctgggaagaagcagccgcacgggggccagcagaagccctcctacggctcgggctaccagtcccaccagggccagcagcagtcctacaaccagagcccctacagcaactatggccctccacagggcaagcagaaaggctataaccatggacaaggcagctactcctactcgaactcctacaactctcccgggggcgggggcggatccgactacaactacgagagcaaattcaactacagtggtagtggaggccgaagcggcgggaacagctacggctcaggcggggcatcctacaacccagggtcacacgggggctacggcggaggttctgggggcggctcctcataccaaggcaaacaaggaggctactcacagtcgaactacaactccccggggtccggccagaactacagtggccctcccagctcctaccagtcctcacaaggcggctatggcagaaacgcagaccacagcatgaactaccagtacagataa

SEQ ID NO: 5 - protein, 702 aa, Homo sapien [sequence omitted]

SEQ ID NO: 6 - DNA, 2107 bp, Homo sapien
atgcgtccaatgcgaatttttgtgaatgatgaccgccatgtgatggcaaagcattcttccgtttatccaacacaagaggagctggaggcagtccagaacatggtgtcccacacggagcgggcgctcaaagctgtgtccgactggatagacgagcaggaaaagggtagcagcgagcaggcagagtccgataacatggatgtgcccccagaggacgacagtaaagaaggggctggggaacagaagacggagcacatgaccagaaccctgcggggagtgatgcgggtgggcctggtggcaaagtgcctcctactcaagggggacttggatctggagctggtgctgctgtgtaaggagaagcccacaaccgccctcctggacaaggtggccgacaacctggccatccagcttgctgctgtaacagaagacaagtacgaaatactgcaatctgtcgacgatgctgcgattgtgataaaaaacacaaaagagcctccattgtccctgaccatccacctgacatcccctgttgtcagagaagaaatggagaaagtattagctggagaaacgctatcagtcaacgaccccccggacgttctggacaggcagaaatgccttgctgccttggcgtccctccgacacgccaagtggttccaggccagagccaacgggctgaagtcttgtgtcattgtgatccgggtcttgagggacctgtgcactcgcgtgcccacctggggtcccctccgaggctggcctctcgagctcctgtgtgagaaatccattggcacggccaacagaccgatgggtgctggcgaggccctgcggagagtgctggagtgcctggcgtcgggcatcgtgatgccagatggttctggcatttatgacccttgtgaaaaagaagccactgatgctattgggcatctagacagacagcaacgggaagatatcacacagagtgcgcagcacgcactgcggctcgctgccttcggccagctccataaagtcctaggcatggaccctctgccttccaagatgcccaagaaaccaaagaatgaaaacccagtggactacaccgttcagatcccaccaagcaccacctatgccattacgcccatgaaacgcccaatggaggaggacggggaggagaagtcgcccagcaaaaagaagaagaagattcagaagaaagaggagaaggcagagcccccccaggctatgaatgccctgatgcggttgaaccagctgaagccagggctgcagtacaagctggtgtcccagactgggcccgtccatgcccccatctttaccatgtctgtggaggttgatggcaattcattcgaggcctctgggccctccaaaaagacggccaagctgcacgtggccgttaaggtgttacaggacatgggcttgccgacgggtgctgaaggcagggactcgagcaagggggaggactcggctgaggagaccgaggcgaagccagcagtggtggcccctgccccagtggtagaagctgtctccacccctagtgcggcctttccctcagatgccactgccgagcaggggccgatcctgacaaagcacggcaagaacccagtcatggagctgaacgagaagaggcgtgggctcaagtacgagctcatctccgagaccgggggcagccacgacaagcgcttcgtcatggaggtcgaagtggatggacagaagttccaaggtgctggttccaacaaaaaggtggcgaaggcctacgctgctcttgctgccctagaaaagcttttccctgacacccctcgcccttgatgccaacaaaaagaagagagccccagtacccgtcagagggggaccgaaatttgctgctaagccacataaccctggcttcggcatgggaggccccatgcacaacgaagtgcccccaccccccaaccttcgagggcggggaagaggcgggagcatccggggacgagggcgcgggcgaggatttggtggcgccaaccatggaggctacatgaatgccggtgctgggtatggaagctatgggtacggaggcaactctgcgacagcaggctacagtgactttttcacagactgctacggctatcatgattttgggtcttcctag

SEQ ID NO: 7 - protein, 406 aa, Homo sapien
MRGDRGRGRGGRFGSRGGPGGGFRPFVPHIPFDFYLCEMAFPRVKPAPDETSFSEALLKRNQDLAPNSAEQASILSLVTKINNVIDNLIVAPGTFEVQIEEVRQVGSYKKGTMTTGHNVADLVVILKILPTLEAVAALGNKVVESLRAQDPSEVLTMLTNETGFEISSSDATVKILITTVPPNLRKLDPELHLDIKVLQSALAAIRHARWFEENASQSTVKVLIRLLKDLRIRFPGFEPLTPWILDLLGHYAVMNNPTRQPLALNVAYRRCLQILAAGLFLPGSVGITDPCESGNFRVHTVMTLEQQDMVCYTAQTLVRILSHGGFRKILGQEGDASYLASEISTWDGVIVTPSEKAYEKPPEKKEGEEEEENTERTTSRRGRRKHGNSGVTFPSLLFLPKGKTGA

SEQ ID NO: 8 - DNA, 1221 bp, Homo sapien
atgaggggtgacagaggccgtggtcgtggtgggcgctttggttccagaggaggcccaggaggagggttcaggccctttgtaccacatatcccatttgacttctatttgtgtgaaatggcctttccccgggtcaagccagcacctgatgagacttccttcagtgaggccttgctgaagaggaaccaggacctggctcccaattctgctgaacaggcatctatcctttctctagtgacaaaaataaacaatgtgattgataatctgattgtggctccagggacatttgaagtgcaaattgaagaagttcgacaggtgggatcctataaaaaggggacaatgactacaggacacaatgtggctgacctggtggtgatactcaagattctgccaacgttggaagctgttgctgccctggggaacaaagtcgtggaaagcctaagagcacaggatccttctgaagttttaaccatgctgaccaacgaaacaggctttgaaatcagttcttctgatgctacagtgaagattctcattacaacagtgccacccaatcttcgaaaactggatccagaactccatttggatatcaaagtattgcagagtgccttagcagccatccgacatgcccgctggttcgaggaaaatgcttctcagtccacagttaaagttctcatcagactactgaaggacttgaggattcgttttcccggctttgagcccctcacaccctggatccttgacctactaggccattatgctgtgatgaacaaccccaccagacagcctttggccctaaacgttgcatacaggcgctgcttgcagattctggctgcaggactgttcctgccaggttcagtgggtatcactgacccctgtgagagtggcaactttagagtacacacagtcatgaccctagaacagcaggacatggtctgctatacagctcagactctcgtccgaatcctctcacatggtggctttaggaagatccttggccaggagggtgatgccagctatcttgcttctgaaatatctacctgggatggagtgatagtaacaccttcagaaaaggcttatgagaagccaccagagaagaaggaaggagaggaagaagaggagaatacagaaagaaccacctcaaggagaggaagaagaaagcatggaaactcaggagtgacattcccttcactccttttcctacccaagggaaagactggagcctaa

(19) United States
(12) Patent Application Publication
(10) Pub. No.: US 2006/0110404 A1
(43) Pub. Date: May 25, 2006

(54) Set of ubiquitous cellular proteins involved in viral life cycle

(75) Inventors: Sven-Erik Behrens, Philadelphia, PA (US); Olaf Isken, Philadelphia, PA (US); Claus W. Grassmann, Bochum (DE); Robert T. Sarisky, Lansdale, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SmithKline Beecham Corporation, Philadelphia, PA (US)

(21) Appl. No.: 10/532,050

(22) PCT Filed: Sep. 12, 2003
(86) PCT No.: PCT/US03/28654

§ 371 Date: Aug. 17, 2005

Related U.S. Application Data
(60) Provisional application No. 60/410,460, filed on Sep. 13, 2002.

(51) Int. Cl. C12Q 1/68; A61K 39/12

(52) U.S. Cl. 424/204.1; 435/6

(57) ABSTRACT

A method of modulating viral RNA replication and translation, in a eukaryotic cell, of positive-strand viral RNA, comprising the step of contacting a viral RNA-binding protein (vRbp) with a compound that modulates an activity of said protein.

### FIELD OF THE INVENTION

This invention relates to newly identified methods for modulating viral RNA replication and translation of positive-strand viral RNA, particularly for the prevention or treatment of viral infections, especially those infections of humans.

### BACKGROUND OF THE INVENTION

A broad spectrum of viruses belonging to the families Picornaviridae, genera Enterovirus, Rhinovirus, Cardiovirus, Aphtovirus and Hepatovirus (hepatitis A virus), and Flaviviridae, genera Flavivirus, Pestivirus and Hepacivirus (hepatitis C virus) are causative agents of wide-spread human and animal diseases (reviewed in 1, 2). For example, pestiviruses such as bovine viral diarrhea virus (BVDV) and classical swine fever virus (CSFV) are pathogens of ruminants and pigs, which cause heavy losses in stock farming. Infection with human Rhinovirus (HRV) represents the main reason for the virus-induced common cold in man. Infection with HCV is a major cause of human liver disease throughout the world with seroprevalence in the general population ranging from 0.3 to 2.2% to as high as ˜10-20% in Egypt. Neither vaccination strategies nor efficient treatments could yet be developed for HRV as well as HCV infections. Accordingly, a major goal in current research on Picornaviridae and Flaviviridae concerns the definition of reasonable targets for antiviral approaches.

The genome of Picornaviridae and Flaviviridae represents a single-stranded, unsegmented RNA molecule of positive polarity. The genome organization is monocistronic, which implies that the RNA consists of a single open reading frame (ORF) flanked by untranslated regions (UTRs) at the 5′ and 3′-end, respectively. Following infection and uncoating, the viral genome operates as a messenger RNA in the cytoplasm of the host cell. Translation leads to the synthesis of an unstable polyprotein that is co- and post-translationally processed by cellular as well as viral proteases to give rise to the virus structural and non-structural proteins. The structural proteins constitute the virus particle: in the case of Picornaviridae, these concern typically four capsid proteins; in the case of Flaviviridae, the virion is composed of a capsid and a membrane envelope, the latter which contains two to three membrane-associated viral envelope proteins. The non-structural proteins, which are predominantly generated by the activity of well-characterized viral proteases, are anticipated or have been demonstrated to act as catalytic components of the viral multiplication machinery. Virus-encoded enzymatic functions, beyond that of the viral proteases, which are essentially involved in the RNA replication process, include an RNA helicase and/or a nucleoside triphosphatase and an RNA-dependent RNA polymerase (RdRp) activity (FIG. 1, see also references 1 and 2).

Translation of the picornaviral as well as of the pestiviral and hepaciviral genomes is controlled by a unique mechanism, which significantly differs from the typeI m7G cap-dependent/ribosome scanning scheme of most eukaryotic messenger RNAs. Extensively structured IRES elements which span a major part of the 5′UTR and in certain cases also the 5′-part of the ORF promote internal entry of ribosomes, i.e. they enable initiation of translation independently of capping and of a free 5′-end (3-10). This strategy allows some viruses to induce a general shut-off of the cap-depending cellular translation while maintaining protein synthesis from their own RNA (reviewed in 11). To support internal translation initiation, these viruses use a basic set of eukaryotic initiation factors but apply some modifications with respect to common mRNAs. Whereas picornaviral IRESes recruit nearly the same set of canonical translation initiation factors as capped mRNAs (12, 13), the HCV and pestivirus type IV IRES elements are capable to form the 40S eIF3 ternary pre-initiation complex autonomously (14). Recent data suggest that a network of interactions of tertiary structure motifs of the HCV core IRES with the 40S ribosomal subunit facilitates the association of the 4S (40S eIF3) particle with the translational start site in the absence of canonical translation initiation factors (15). The exact mechanism by which IRES elements mediate translation initiation remains to be determined. In addition to the canonical initiation factors (Ifs), the diverse IRES elements were found to bind other cellular proteins, which are suspected or have been shown to enhance translation efficiency, to confer tissue specificity or to mediate the regulation between translation of the infecting RNA and its replication (see below). In agreement with this concept, proteins such as La, poly C binding protein (PCBP) or hnRNP E, and poliovirus translation factor (PTF), polypyrimidine tract binding protein (PTB) or hnRNP I, have been associated with the translation of Enteroviruses; PTB and unr/unrip with HRV; PTB with Aphtoviruses; Liver-specific factors, GAPDH (glyceraldehyde 3-phosphate dehydrogenase) and PCBP with HAV; and PTB, PCBP, the ribosomal proteins S9 and L22, La, and hnRNP protein L with HCV (reviewed in references 16-18, for the non-reviewed data see 19-21).

The intracellular multiplication of the viral RNA occurs as a Two-step process, the molecular mechanisms of which are far from being understood (see references 1, 2, and FIG. 1). Although the priming mechanisms to initiate the synthesis of novel RNA molecules ought to be different in Picornaviridae and Flaviviridae some general homologies exist. RNA replication is known to occur exclusively in the cytoplasm of the host cell and to proceed asymmetrically along a two-step pathway. Concomitant with translation and proteolysis of the polyprotein, a set of non-structural viral proteins is presumed to associate with the termini of the genome to form membrane-associated replication complexes. The replication complexes initially catalyze transcription of a small number of complementary negative-strand RNA intermediates from which, in turn, an excess of progeny positive-strand RNA molecules are generated. Several lines of evidence suggest that Picornaviridae as well as Flaviviridae subvert cellular factors (host-factors) to participate as functional components of their replication complexes: to confer, for example, template RNA specificity to the RdRp (which is not present in vitro) or to mediate the transition between translation and RNA replication. In this context, cellular factors are discussed, which have been found to interact with the 3′UTR of poliovirus (nucleolin, see reference 22), flaviviruses (eIF1α, see reference 23), or of HCV (PTB, HuR, hnRNP C; see review 18 and references 24, 25).

As a common feature of the life cycle of all monocistronic positive-strand RNA viruses, FIG. 1, the viral genome has to exert two essential functions in the cytoplasm of the infected host-cell. On the one hand, the RNA is translated in 5′-3′ direction, on the other hand, it acts as a template for the viral RdRp, which is expected to initiate the replication cycle at the 3′-end of the genomic RNA moving 3′ to 5′. The mechanisms of how the RNA switches between both interdependent, although possibly competing processes is unknown but they are essential for the regulation of the overall virus life cycle. Data, which emerged mainly from studies with picornaviruses (reviewed in reference 26) suggest the following model. During the mRNA phase, translation prevents the initiation of the replication cycle. Then, at a certain stage, the initiation of translation is blocked causing the release of ribosomes from the viral RNA. Finally, formation or activation of the initial replication complex “locks” the viral RNA into a replication mode and promotes the synthesis of negative-strand RNA. A reasonable model to explain the transition from translation to RNA replication, and possibly vice versa, is a feedback communication of the UTRs of the viral genome involving the viral replication complex on the one hand and cellular host factors on the other hand. The latter proteins are expected to be associated with the translation machinery but to interact also with viral proteins and/or regulatory elements of the viral RNA. Such a model suggests a functional cross-talking between 5′ and the 3′-end of the viral RNA—similarly as it has been proposed during translation regulation of capped eukaryotic mRNAs (27).

Preliminary data from the poliovirus system suggest that translation takes place until an adequate quantity of the viral polypetide 3CD^{pro} is accumulated (28). Aided by the host factor PCBP1, 3CD^{pro} then interacts with a certain RNA-structure, cloverleaf, at the immediate 5′-end of the genome. This motif is essentially involved in both steps of the RNA replication process. Moreover, it modulates the IRES-mediated translation process (29). The viral/cellular ribonucleoprotein (RNP) complex is suspected to repress translation and to promote negative-strand RNA synthesis (28). Interestingly, 3CD^{pro} was shown to associate with poly(A)⁺ binding protein (pAb1p) (30). As a possible scenario, pAB1p might contact an A-rich region in the 3′UTR and thus could bring about a functional 5′-3′ interaction of the poliovirus genome. Data obtained with atomic force microscopy indicate indeed a closed loop conformation of the poliovirus genome (31). Indications for a 5′-3′ communication of the viral genome exist also for the flavivirus Kunjin and hepatitis C virus (32, 33).

The identification of cellular factors or vRbps, which are critical for the intracellular multiplication process of RNA viruses, and the characterization of the functional interplay between these factors with viral proteins and genomic elements of the viral RNA are key to understanding replication of these viruses. Inhibiting the biological activity of such factors may potentially benefit cells by controlling, reducing and alleviating diseases caused by infection with these viruses.

Many viruses encode protein factors to circumvent the antiviral response of the cellular host to an infection. Along this line, certain viral proteins such as the vaccinia E3L, the influenza virus protein NS1 and the rotavirus NSP3 associate with double-stranded (ds) RNA, and bind to dsRNA-dependent protein kinase (PKR) in order to inhibit its antiviral activity (reviewed in reference 34). PKR, the expression of which is induced by dsRNA and/or the activity of interferons, e.g., as a result of a viral infection, is a serine/threonine kinase with multiple functions in control of transcription and translation (reviewed in reference 35). The enzyme, which is activated through its binding to dsRNA, plays a role in mediating apoptosis as well as signal transduction events that are involved in the interferon response of the cell to accelerate virus clearance. Moreover, the activated PKR phosphorylates the a subunit of the eukaryotic translation initiation factor eIF2. Phosphorylation of eIF2α inhibits the recycling of eIF2 and consequently blocks the cellular translation machinery in response to viral infection. Accordingly, proteins, which mimic the PKR-eIF2α interaction domain, were found to inhibit the activity of PKR (34).

### SUMMARY OF THE INVENTION

The invention relates to a set of cellular polypeptides, their production and uses, as well as variants, agonists and antagonists and their uses. In particular, in these and in other regards, the invention relates to a set of cellular polypeptides, hereinafter referred to as viral RNA binding proteins (vRbp). The set of cellular polypeptides preferably associate with the untranslated regions of the genomes of different representatives of virus families, preferably, the Picornaviridae and Flaviviridae families. The experimental data obtained with the Flaviviridae members BVDV and HCV implicate these proteins are involved in the regulation of the translation and replication process of the viral RNA. Preferably, they may be crucially involved in the regulation of the translation and replication process of the viral RNA. Remarkably, the majority of these cellular polypeptides represent dsRNA binding proteins, which may associate with PKR and thus inhibit its activity. Therefore, the recruitment of these factors by the diverse viral RNAs may serve a second purpose, i.e., to block the antiviral activity of PKR in the host cell. The newly identified viral/cellular ribonucleoprotein (RNP) complex is accordingly expected to represent a meaningful target for antiviral substances that are either capable to interfere directly with the viral multiplication process or to increase the efficiency of the endogenous antiviral response.

One aspect of the invention is a method for modulating viral RNA replication and translation, in a eukaryotic cell, of positive-strand viral RNA, comprising the step of contacting a viral RNA-binding protein (vRbp) with a compound that modulates an activity of said vRbp. Preferred aspects of this method include vRbps selected from the group consisting of: vRbp130, vRbp120, vRbp110, vRbp84, vRbp64, and vRbp45. In other alternative methods, the activity of the vRbp is selected from the group consisting of a response to viral RNA, interferon induction, double-stranded RNA-dependent protein kinase (PKR), and to another vRbp. Furthermore, other embodiments of the claimed invention include a response to the formation of a viral:cellular ribonucleoprotein (RNP) complex. Alternative RNP complexes include a viral RNA:vRbp interaction, binding of a vRbp to a viral RNA 3′ untranslated region (3′UTR) or binding of a vRbp to a viral RNA 5′ untranslated region (5′UTR). Another embodiment of the invention is wherein the 3′UTR is a UGA box consensus sequence.

In still another aspect of the invention, methods for modulating viral RNA replication and translation include modulating the activity of a vRbp wherein the activity is a response to viral RNA circularization. In one aspect of the invention includes modulating the binding of vRbp to the viral 5′UTR and 3′UTR, which creates a physical and functional link between both ends of the RNA. A preferred embodiment of the invention provides for a method of modulation an interaction between viral 5′UTR, 3′UTR RNA, vRbp, and cellular proteins involved in the interferon antiviral response.

In yet another aspect of the invention, methods for modulating viral RNA replication and translation include modulating the activity of a vRbp wherein the activity is a response to an increase in translational frameshifting that result in decreased viral replication, or formation of a vRbp:PKR interaction.

Other embodiments of the invention include methods for modulating viral RNA replication and translation wherein viral replication and translation comprises coordinated regulation of replication and translation of viral RNA.

Alternative embodiments include methods for modulating viral RNA replication and translation wherein the eukaryotic cell is, but not limited to, a mammalian cell, a human cell, or a liver cell.

Alternative embodiments include methods for modulating viral RNA replication and translation wherein viral RNA is positive strand viral RNA from viral families including Flaviviridae and Picornaviridae.

Other aspects of the present invention include compounds for modulating viral RNA replication and translation. Alternative embodiments include therapeutically effective amounts of viral 3′UTR, fragments thereof, or pharmaceutically acceptable derivatives thereof for modulating viral RNA replication and translation. Further embodiments of the invention include methods for reducing vRbp activity by interfering with the interaction between vRbp and vRbp recognition sites on viral RNA. One embodiment that reduces vRbp activity is by modification of a viral 3′UTR, which modification otherwise reduces vRbp binding to vRbp recognition sites on viral RNA. Another embodiment that reduces vRbp activity is by inhibiting dissociation of viral RNA:vRbp complexes.

In another aspects of the invention, method for reducing the effects of viral infection on eukaryotic cells, comprising inhibiting vRbp activity in the cell such that viral replication and translation of viral RNA is regulated by interactions between vRbp and said viral RNA, comprising introducing a nucleic acid decoy molecule into the cell in an amount sufficient to inhibit viral RNA:vRbp interactions, which decoy includes a vRbp recognition site that binds to vRbp. Alternative methods for reducing the effects of viral infection on eukaryotic cells, include inhibiting vRbp activity in the cell such that vital replication and translation of viral RNA is regulated by interactions between vRbp and PKR, comprising introducing a nucleic acid decoy molecule into the cell in an amount sufficient to inhibit vRbp:PKR interactions, which decoy includes a vRbp recognition site that binds to vRbp.

Additional aspects of the invention include methods for reducing the effects of viral infection on eukaryotic cells, comprising the step of reducing vRbp activity in the cell such that viral replication and translation is reduced. Prefered embodiments include methods for reducing the effects of viral infection on eukaryotic cells, the method comprising the step of reducing vRbp activity in the cell such that production of novel infectious virus particles is reduced, steps of reducing vRbp activity in the cell to inhibit the spread of virus in infected individuals and animals, steps of reducing vRbp activity in the cell to prevent the spread of virus between different individuals and animals, or steps of reducing vRbp activity in the cell to treat syndromes caused by co-infection of different viruses, such as, HCV and HBV or HCV and HIV. Othere alternatives to methods reducing the effects of viral infection on eukaryotic cells, include steps of reducing vRbp activity in the cell to treat before, during, and after a transplantation, steps of modulating vRbp activity in the cell to treat immunosuppressed patients to prevent virus infections.

Another aspect of the invention includes a method for reducing the effects of viral infection, in a eukaryotic cell, by modulating vRbp activity in the cell, the method comprising the step of interfering with viral translation termination as a mechanism to disrupt viral replication. Furthermore, an alternative method of the invention for reducing the effects of viral infection, in a eukaryotic cell, is to modulate viral RNA-binding protein (vRbp) activity in the cell, the method comprising the step of interfering with interactions between viral 3′UTR and 5′UTR , or interactions between structural elements within the 3′UTR and NS5B stop codon as a mechanism to regulate translation termination, translational frameshifting, and the coordinated balance of replication and translation on positive strand RNA, such as RNA from a member of the family Flaviviridae, or Picornaviridae.

Other embodiments of the invention include a method of treating or preventing a viral infection by a virus comprising the step of administering a therapeutically effective amount of a compound to an individual suspected of having or being at risk of having an infection with a virus, such as, hepatitis A virus (HAV), hepatitis C virus (HCV), human Rhinovirus (HRV), bovine viral diarrhea virus (BVDV), and classical swine fever virus (CSFV). An embodiment of the claimed compound may compound interact with viral genomic 3′UTR or 5′UTR RNA. Alterative aspects of the invention include methods for modulating the function of a viral 3′UTR comprising the step of contacting a 3′UTR with a compound that modulates the structure of the 3′UTR as to inhibit the interaction between 3′UTR and vRbp.

Another aspect of the invention is a method for screening to identify compounds that activate or that inhibit the function of vRbp which comprises a method selected from the group consisting of:
(a) mixing a candidate compound with a solution containing a vRbp, to form a mixture, measuring activity of the vRbp in the mixture, and comparing the activity of the mixture to a standard;
(b) detecting the effect of a candidate compound on the production of viral RNA in a eukaryotic cell, using for instance, an ELISA assay, reticulocyte lysate translation assay (luciferase RNA); and
(c) (1) contacting a composition comprising the vRbp with the compound to be screened under conditions to permit interaction between the compound and the vRbp to assess the interaction of a compound, such interaction being associated with a second component capable of providing a detectable signal in response to the interaction of the vRbp with the compound; and
   (2) determining whether the compound interacts with and activates or inhibits an activity of the vRbp by detecting the presence or absence of a signal generated from the interaction of the compound with the vRbp.

An alternative embodiment of the invention is a method for screening to identify compounds that increase translational frameshifting resulting in decreased replication of viral RNA comprising a method selected from the group consisting of:
(a) mixing a candidate compound with a solution containing a vRbp, to form a mixture, measuring activity of the vRbp in the mixture, and comparing the activity of the mixture to a standard; and
(b) detecting the effect of a candidate compound on the production of viral RNA in a eukaryotic cell, using for instance, an ELISA assay, reticulocyte lysate translation assay (luciferase RNA).

Other aspects and advantages of the present invention are described further in the following detailed description of the preferred embodiments thereof.

### BRIEF DESCRIPTION OF THE FIGURES

FIG. 1 graphically illustrates the genome organization and replication cycle of Picornaviridae, Pestiviruses and Hepacivirus. (A) Schematic representation of the organization of Picornaviridae, Pestiviruses and Hepatitis C virus genomes. The 5′ and 3′untranslated regions (UTRs) are indicated as black lines, the protein-coding region (ORF) as a box. The proteolytic cleavage products of the ORF-encoded polyprotein are shown as differently shaded regions. The dot at the 5′-end of the Picornaviridae genome indicates the VPg protein (or 3B protein), which is associated to the 5′-end of all Picornaviridae RNAs. L specifies a leader protein found in cardioviruses, Theiler viruses and aphtoviruses; it is not present in enteroviruses , human rhinovirus, or human hepatitis A virus. 1A-1D represent the Picornaviridae capsid proteins. C, E^{RNS}, E1 and E2 are the structural components of the Pestivirus virion. C, E1 and E2 are the structural components of the Hepacivirus. Note that Picornaviruses have different internal ribosomal entry sites (types I-III). The IRES of Pestiviruses and Hepacivirus was termed as type IV. (B) Schematic representation of the replication pathway of monocistronic RNA viruses. Upper level: general organization of the genome of monocistronic positive-strand RNA viruses (see A). The 5′-end may be either capped (as with Flaviviruses) or it may contain an IRES region, the 3′UTR may be polyadenylated or not. For a detailed description of the replication scheme see text or references 1 and 2.

FIG. 2 graphically illustrates organization of monocistronic and bicistronic BVDV and HCV RNA replicons. Top: organization of subgenonic BVDV replicon RNAs in comparison with the full-length viral genome. In the case of the monocistronic BVDV replicon “DI9c,” the coding region of the pestiviral protein N^{pro} is directly fused to the NS3 coding region. N^{pro} is an autoprotease and enables the generation of the NS3 protein with its authentic N-terminus. DI9c or functional parts of it have been used in most experiments, which were aimed at characterizing the different functional determinants of the translation and replication process of the BVDV RNA (see text). “Bicistronic replicons” contain an additional, heterologous ORF. The additional gene may encode a resistance-marker (Hyg=hygromycine B phosphotransferase; Neo=neomycin phosphotransferase) or other enzymes (e.g. GUS=β-glucoronidase). The additional ORF was cloned upstream of an encephalomyocarditis (EMCV) IRES-element, the latter which maintains expression of the viral non-structural proteins. Generation of the authentic N-terminus of the heterologous gene product was enabled by fusing a portion of the N^{pro} gene, this is necessary to ascertain efficient IRES function, and an ubiquitine gene to the 5′-terminus of the additional ORF. Generation of the authentic N-terminus of the heterologous protein is thus enabled by the activity of cellular ubiquitin C-terminal hydrolases. Two types of BVDV replicons were employed in our assay-systems, namely ncp and cp types. Ncp implies that these RNase are non-cytopathic and hence persist in the transfected host-cell. These RNAs express predominantly the full-length NS2-3 protein. Generation of the authentic N-terminus of NS2-3 is enabled by cellular peptidases, which cleave at the C-terminus of the peptide p7, which is also encoded by the ORF. Cp indicates cytopathogenicity, i.e., lysis of the host-cell at a certain time post transfection. A cytopathogenic phenotype correlates with the predominant expression of NS3 (2). Accordingly, DI9c represents a cp replicon RNA. Bottom: organization of mono and bicistronic HCV replicons. The organization basically resembles to that of the BVDV replicons described above. ΔC indicates a short region of the Core protein-coding region, which was shown to be important for efficient translation initiation. In certain cases, a ubiquitine gene was inserted.

Abbreviations: mono-monocistronic, bi-bicistronic, cp-cytopathic, ncp-non cytopathic, Δ-indicates an incomplete genetic unit, ubi-indicates the ubiquitine gene to mediate proteolytic cleavage by ubiquitine carboxy-terminal hydrolases at this position), het. gene-indicates a gene encoding a heterologous protein. The proteolytic cleavage sites are indicated as follows: arrow-cleavage by NS3/NS4A, circle-cleavage by cellular signalases, A-autoproteolytic activity, ?-uncertain.

FIG. 3 graphically illustrates RNA secondary structure of the 3′UTRs of a BVDV (strain CP7/CP9; see 36 and references herein) and of an HCV isolate (strain 1B; 38). The depicted sequence initiates with the translational UGA stop-codon (indicated by italics). The structure of the BVDV 3′UTR was determined by experimental means (43): nucleotide residues that were found to be exposed to RNases or chemical modification are indicated in dark grey (highly exposed) or light grey (less exposed). The UGA box elements and pseudo-stops are boxed. The arrow marks the border between the 3′V and 3′C regions as proposed by Deng and Brock (47). The RNA secondary structure of the HCV 3′UTR was calculated with the mfold 3.1 computer program.

FIG. 4 graphically illustrates (A) Secondary structure of the 5′UTRs of BVDV and HCV (reviewed in reference 16). The diverse RNA domains and the AUG translational start-codon are indicated. The minimal IRES elements are boxed, the so called “core-domains” are marked by dashed circles. HCV 5′UTR: the arrows indicate regions, which were found to harbour important replication signals (52, 44). (B) Structure and functions of the BVDV hairpin Ia and “hairpin Ib” motifs. The structures of Ia and Ib were determined by Yu et al. (43, 45): residues that were found to be exposed to RNases or chemical modification are indicated as in FIG. 3. Hairpin Ib is written in quotation marks, because the experimental data contradict the formation of a hairpin structure. Nucleotides that are essential for replication are boxed; elements that enhance the replication efficiency are indicated by dashed boxes. Elements that enhance the translation efficiency are indicated by a dashed circle (43, 45).

FIG. 5 graphically illustrates (A) A set of cellular proteins binds to the 3′UTR of the BVDV DI9c replicon RNA. UV cross-linking/label transfer experiments were performed with viral and non-viral RNA probes and cytoplasmic extracts of BHK-21 cells. The composition of the utilized RNA probes is schematized in the lower part of the figure. Indicated are the restriction sites that were used to generate the respective templates for run-off transcription, and, in the case of the viral RNAs (3′BVDV and 3′HCV), the translational stop-codon. A grey box depicts the non-related BKS RNA; open boxes correspond to the untranslated regions of the viral RNAs, black boxes stand for residual parts of the viral ORF. Cytoplasmic extracts (total amount of protein: ca. 20 μg/assay volume) of mock-transfected, lanes 1, 3, 5 and 7, or BVDV DI9c transfected BHK-21 cells, lanes 2, 4, 6 and 8, were utilized for cross-linking with the different [³²P] UTP-labeled RNA transcripts. Protein labeling was analyzed by 10% SDS-PAGE. In the control-reactions shown in lane 7 and 8, RNA-protein complexes formed on radio-labeled 3′BVDV RNA were digested with proteinase K prior to exposure to UV-light. Marker proteins are indicated on the left; the most significantly RNA-charged proteins, marked by arrows, were denoted according to their suggested molecular weights, namely p130, p120, p110, p84, p67 p64, and p45 (termed as “vRbps” in the text). (B) The same set of RNA-binding proteins is present in various cell types and interacts with the 3′UTR of different pestiviruses. Top: cross-linking study with labeled BVDV DI9c 3′UTR and cytoplasmic extracts of BHK-21, MDBK and HeLa S3 cells. UV cross-linking/label transfer was performed with BKS RNA, lanes 1 to 3, or 3′BVDV RNA, lanes 4 to 6), respectively. Positions of the most strikingly labeled proteins are indicated by arrows, see FIG. 5A. By competition experiments, see FIG. 5C, the protein marked with an asterisk, lane 6, was demonstrated to bind non-specifically to the viral RNA (data not shown). Bottom: cross-linking study with labeled BVDV or CSFV 3′UTR RNA using cytoplasmic extract of BHK-21 cells (similar results were obtained with extracts of other cell types, data not shown). The composition of the different RNA probes is schematized in the lower part of the figure. Lane 1, control assay with BKS RNA; lane 2, cross-link assay with 3′BVDV RNA; lane 3,˜with 3′CSFV RNA; lane 4, control reaction with 3′BVDV RNA, performed as described in FIG. 5A. Molecular weights are indicated on the left. Arrows point at the major RNA-protein complexes. (C) Cellular proteins p130, p120, p110, p84, p64, and p45 bind in a specific manner to the pestiviral 3′UTR. Aliquots of cytoplasmic extract of BHK-21 cells, approximately 10 μg of total protein/assay, were incubated with either [³²P]-labeled BKS RNA probe, lanes 1 to 3, or 3′BVDV RNA, lanes 4 to 8, in the absence or presence of the below-indicated amounts of unlabeled competitor RNA, respectively. After treatment with UV-light, the proteins were analyzed on SDS-PAGE. Lane 1 and 4, assay without competitor; lane 2 and 5, identical experiment performed as in lane 1 but in the presence of a 200 fold molar excess of non-specific BKS competitor RNA; lane 3 and 6,˜in the presence of a 200 fold molar excess of specific 3′BVDV competitor RNA; lane 7,˜in the presence of a 200 fold molar excess of 3′CSFV RNA; lane 8,˜in the presence of a 200 fold molar excess of 3′HCV RNA. As in the previous figures, the molecular masses of the radiolabeled ribonucleoprotein complexes are indicated by arrows. (D) Exploring the BVDV DI9c 3′UTR for the host factor binding site(s). Comparison of 3′V regions of different pestivirus genotypes, sequences obtained from Genbank databases, revealed the conservation of stretches of 12 nucleotides. The nucleotide sequences of representatives of the different pestivirus genotypes (BVDV-1, BVDV-2, CSFV and BDV) were extracted from the GenBank/EMBL database and computer-aligned. An A/U-rich sequence element, which is present in all different viral genomes was found to be located at either position 43 or 46 of the respective 3′UTR; it was termed UGA_{pos.cons.}box (nomenclature as in FIG. 3). Interestingly, most of these motifs (only exception BVDV NADL) are positioned “in frame” with the viral ORF. Hence, the distance of the UGA_{pos.cons.} box with regard to the translational stop codon corresponds to 14 or 15 triplet-units, “pseudo codons,” respectively. As indicated by the consensus sequence shown in the lower part of the figure, the UGA_{pos.cons.} boxes contain 4 nucleotides that are 100% conserved, (bold typed and underlined) among all different viral genomes. These nucleotides are also conserved in other, “additional” UGA boxes such as those of BVDV Osloss, BVDV CP7 (BVDV DI9c) and BVDV Singer at position 16 or 19 of the respective 3′UTR. Note that most UGA boxes contain “pseudo stop-codons” such as UAA at their 3′-end. (E) p130, p120, p110, p84, p64, and p45 bind specifically to a single UGA box sequence motif. Left: UV-induced label-transfer experiments with cytoplasmic extracts of BHK-21 cells and RNA probes containing defined parts of the 3′V region of BVDV DI9c RNA. The composition of the applied RNA transcripts is schematically drawn in the lower part of the figure: Bm1/m2 RNA covers the 5′-terminal part of the BVDV DI9c 3′V region (residues 10-63 in the numbering scheme of FIG. 3) and thus includes the 5′UGA box and the UGA_{pos.cons.} box, depicted as grey boxes. Bm2 RNA consists mainly of the UGA_{pos.cons.} box sequence, grey box, of the BVDV DI9c RNA (residues 40-62 in the nomenclature of FIG. 3). To allow an estimation of the binding capacity of the different RNAs identical molar amounts of Bm1/m2 RNA and Bm2 RNA were employed in the UV cross-linking assay. Lane 1, negative control assay with non-related BKS RNA; lane 2, positive control assay with 3′BVDV RNA; lane 3, assay with Bm1/m2 RNA; lane 4, assay with Bm2 RNA. Molecular weights and positions of the RNA-charged proteins are indicated as in all previous figures. Proteins, which were found to bind non-specifically to the RNA transcripts, data not shown, are marked with asterisks. Right: competition experiments with Bm1/m2 RNA and Bm2 RNA. Competition of the binding of the cellular proteins to 3′BVDV RNA, lanes 1-5, or Bm1/m2 RNA, lanes 6-10, was investigated by using BKS RNA as a non-specific competitor, lanes 2 and 7, and 3′BVDV RNA, lanes 3 and 8, Bm1/m2 RNA, lanes 4 and 9, and Bm2 RNA, lanes 5 and 10, as specific competitors, respectively. To allow an estimation of the competition efficiency of each of the different RNAs, identical molar amounts were included into the respective experiments. Molecular weight markers and positions of RNA-protein complexes are indicated as in the previous figures.

FIG. 6 graphically illustrates Binding of the vRbps to the BVDV 3′V region correlates with the efficiency of translation initation, translation termination, and replication of the viral RNA. (A) RNA secondary structure of the wt BVDV 3′UTR and of two 3′V mutants. The RNA structure was determined by experimental probing (see FIG. 3). Mutant 1 comprised a deletion of 57 residues, i.e., of both 5′-terminal UGA boxes, and a double point-mutation affecting the 3′UGA-like box and the folding of SLII, respectively. Mutant 2 comprised nine point mutations that modified the consensus of all three UGA-boxes, the pseudo-stops and the folding of SIₛₜₒₚ and SLII, respectively. (B) Effect of mutagenesis on the association of the viral RNA binding proteins to the BVDV 3′UTR. Wt and mutant 3′UTRs were tested by UV crosslinking/label transfer for the association of host-factors p130, p120, p110, p84, p67, and p64, respectively. As shown, both mutant RNAs associate the cellular proteins to a significantly lower degree with respect to the wild-type RNA, for further details, see FIG. 5 and text. (C) Effect of mutagenesis on the rate of replication and translation of the viral RNA. Replication was determined with monocistronic BVDV constructs (FIG. 2) via quantitative RNase protection of progeny positive-strand RNA. Translation was quantitated in vitro by the expression of the N^{pro} protein essentially as described by Yu et al. (43). (D) Translational read-through assay. In vitro translation was performed in the presence of [³⁵S] methionine with a minigenomic RNA encoding the UTRs and a shortened ORF (53). With the wt, only the ORF-encoded proteins, NS fus and N^{pro}, the latter which is autoproteolytically released, are expressed. An additional product corresponding in size exactly to the NS fus+translated 3′UTR is detectable with both 3′V mutants (53).

FIG. 7 graphically illustrates data that support the idea of a protein-mediated interaction of the termini of the BVDV RNA. (A) UV crosslinking/label transfer experiments with transcripts of the BVDV 5′UTR, HCV 5′UTR and BVDV 3′UTR. The proteins, which were confirmed to associate specifically to the viral RNAs, see FIG. 5, are indicated as in the previous figures. Asterisks mark proteins found to bind non-specifically. Polypyrimidine-tract binding protein (PTB) is indicated, which was previously shown by the same assay to bind to the HCV 5′UTR. (B) 5′-3′ co-precipitation assay. The experiment was generally performed with a biotinylated 5′UTR transcript and a [³²P]-labelled 3′UTR transcript. Precipitation was performed with streptavidine-beads. As a control, the precipitation was carried out in the absence of protein or in the presence of bovine serum albumine. Note that the data indicate a slight interaction of both termini also in the absence of the cellular proteins. (C) Model of a protein-mediated cross talk of the 5′ and the 3′ end of the viral RNA. 5′-3′ interaction might be a way to coordinate the translation (5′-3′) and the replication (3′-5′) cycle.

FIG. 8 graphically illustrates that different viral IRES elements recruit the same set of cellular proteins. (A) UV crosslinking/label transfer experiments with transcripts comprising the BVDV 5′UTR, BVDV 3′UTR, HAV 5′UTR, EMCV 5′UTR and Rhinovirus 5′UTR. BKS RNA was used as a control. Proteins, which were confirmed to associate specifically to the viral RNAs (see FIG. 5), are indicated as in the previous figures. Asterisks mark proteins that bind non-specifically. Polypyrimidine-tract binding protein (PTB) and unr are indicated, unr was not confirmed. PTB was previously shown by the same assay to bind to the HAV, EMCV and rhinovirus 5′UTR; unr was previously shown to bind to the rhinovirus 5′UTR (1). (B) UV crosslinking/label transfer experiments with transcripts comprising the BVDV 5′UTR, BVDV 3′UTR, HCV 5′UTR and the HCV 5′UTR+GUAU. The experiment was performed as described in the previous figures and in the text. Additional details concerning HCV 5′UTR+GUAU see FIG. 12.

FIG. 9 graphically illustrates data indicating an association of the cellular proteins with the HAV core-IRES domain. The different graphs show the RNA secondary structure of the different core-IRES domains of type I, type II, type III and type IV IRESes as proposed by Le et al. (59). The translational start-codon as well as nucleotides that are 100% conserved between all different viruses are indicated; N stands for a variant number of nucleotides. In the case of the HCV IRES, the core-IRES model exhibits striking similarities with the RNA structure determined by RNase digestion and chemical modification procedures (see 15 and 16 and references herein; and FIG. 4). In comparison with the BVDV and HCV IRES, the HAV IRES element is bigger in size (ca. 350 nt versus 723 nt), and it has a less compact shape (see reference 16). Thus, as a reasonable approach to generate RNA transcripts encompassing the correctly folded core-IRES domain, RNA transcripts corresponding to the HAV 5′UTR were digested with RNaseH in the presence of a suitable oligonucleotide, the site where RNaseH cuts is indicated in the figure. The resulting core-IRES RNA was purified and subjected to a UV-crosslinking/label transfer approach. The pattern of labelled proteins was compared side-by-side with that obtained with full-length HAV 5′UTR and BVDV 3′UTR, respectively. As shown on the right portion of the figure, the pattern of labelled proteins turned out to be nearly identical in all three experiments.

FIG. 10 graphically illustrates purification and identification of the viral RNA binding cellular factors. (A) Purification. Top: scheme summarizing the different fractionation steps, starting material S10 extracts of Hela cells. Bottom fractions of proteins eluted by a salt gradient from the MonoQ sepharose column were tested via UV crosslinking/label transfer assay with 3′BVDV RNA to monitor the elution of the different vRbps. The SDS PAGE shows analysed fractions eluted between 300 and 450 mM KCl, indications as in the previous figures. Lane 1—pattern of labelled proteins obtained by UV crosslinking of total cytoplasmic extract of Hela cells; lane 2—pattern of labelled proteins obtained by UV crosslinking of the heparine flow-thru fraction. Due to the fact that the entire set of proteins elutes in the fractions analysed on lanes 8-10, these fractions were pooled and the proteins separated on a preparative SDS PAGE. Coomassie-stained protein bands migrating at 84 kDa were cut out, digested with trypsine and the peptides extracted from the gel. MALDI-TOF analysis and microsequencing was performed on different tryptic peptides. (B) Identification of the viral RNA binding proteins part I. Top: side-by-side comparison of the pattern of vRbps labelled by UV cross-link/label transfer with radioactive 3′BVDV RNA and the pattern of proteins stained by a mixture of αNF90 and αNF45 antibodies (61) on western blots of total cytoplasmic extracts of Hela cells. The proteins, which are stained by the individual αNF90 and αNF45 antisera are indicated (data of separate blots not shown). The identity of the different proteins was concluded by the results obtained during microsequencing and data that were published on NFAT/NF90/NFAR-1, NFAR2 and NF45 by other laboratories (see text). Bottom: schematic representation of the structure and the motifs harboured by the aminoterminal 686 AA of all members of the NFAT/NF90/NFAR family. Abbreviations: NLS=nuclear localization sequence, dsRBM=double-strand RNA binding motif, RG=arginine glycine-rich RNA binding domain.

FIG. 11 graphically illustrates identification of the viral RNA binding proteins part II. (A) “Supershift” of RNA-protein complexes by αNF90 and αNF45 antibodies. RNA mobility shift assay (RMSA) with [³²P] labelled RNA transcripts. Different amounts of cytoplasmic extracts, increasing amounts from right to left, were incubated with a specific [³²P] labelled RNA probe, e.g., HCV 5′UTR, BVDV 3′UTR, and comparable amounts of a non-specific antiserum and of αNF90 and αNF45 antisera, respectively. The RNP and RNP/antibody complexes (indicated on the right) were separated on a 5% acrylamide/Tris borate gel. (B) RNA-protein coprecipitation (pull-down) assay with in vitro translated NF90 protein. In vitro translated [³⁵S] labelled NF90 protein was incubated with a specific, e.g., HCV 5′UTR, and a non-specific, e.g., BKS RNA transcript, respectively. The unlabelled RNA transcripts contained a poly-A tail and were subsequently precipitated by oligo dT sepharose. In vitro translated [³⁵S] labelled luciferase protein was used as a control.

FIG. 12 graphically illustrates implications for HCV. (A) Schematic representation of functional HCV/BVDV and BVDV/HCV chimeric RNAs. Top/left: RNA secondary structure of hairpin Ia of the BVDV 5′UTR (see also FIG. 4); the four GUAU nucleotides, which were found to be essential for BVDV RNA replication are depicted in red. Top/middle: RNA secondary structure of the HCV hairpin Ia+5′GUAU. BVDV RNA, where the BVDV 5′UTR was substituted by the HCV 5′UTR+GUAU was found to be replication competent (without GUAU, the BVDV RNA was replication deficient, 51). Top/right: UV crosslinking/label transfer analysis of RNA transcripts encompassing the HCV5′UTR, HCV5′UTR+GUAU and the BVDV 5′UTR. The viral RNA binding proteins are indicated as in the previous figures. Bottom/left: schematic drawing of the organization of the hybrid HCV 3′V region containing the BVDV UGA box elements instead of SLₛₜₒₚ, for additional details, see FIG. 12B. Bottom/right: UV crosslinking/label transfer analysis of RNA transcripts comprising the HCV 3′UTR and the HCV 3′UTRΔSLstop+BVDV 5′UGA boxes, respectively. (B) Structure and organization of HCV 3′V mutant RNAs. Top: RNA secondary structure of a BVDV and HCV 3′UTR (see also FIG. 3). Bottom: RNA secondary structure, calculated by mfold 3.1, of the HCV 3′V ΔSLₛₜₒₚ mutant and of the HCV/BVDV 3′V chimera (see FIG. 12A). In FIG. 12B, the BVDV-derived sequence is depicted in light gray (HCV/BVDV chimera 5′ loop); six additional nucleotides corresponding to an Afl1 restriction site in the original cDNA construct are depicted as CUUAAG in the HCV/BVDV chimera FIG. 12B.

FIG. 13 graphically illustrates an RNAi approach with aRHA oligonucleotides inhibits HCV replication.

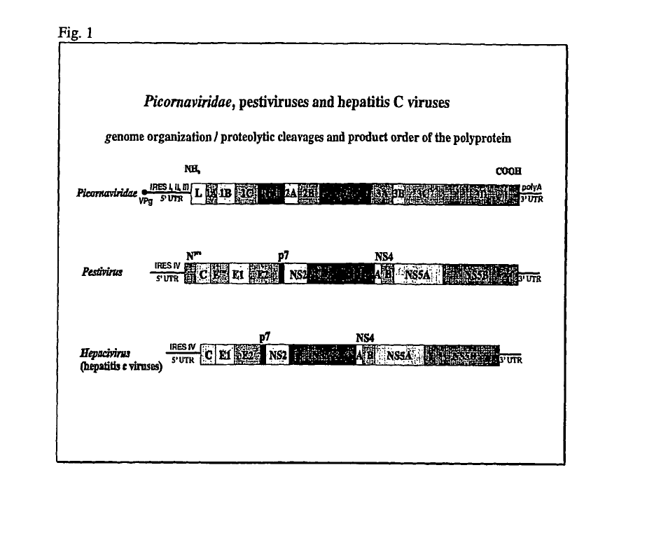

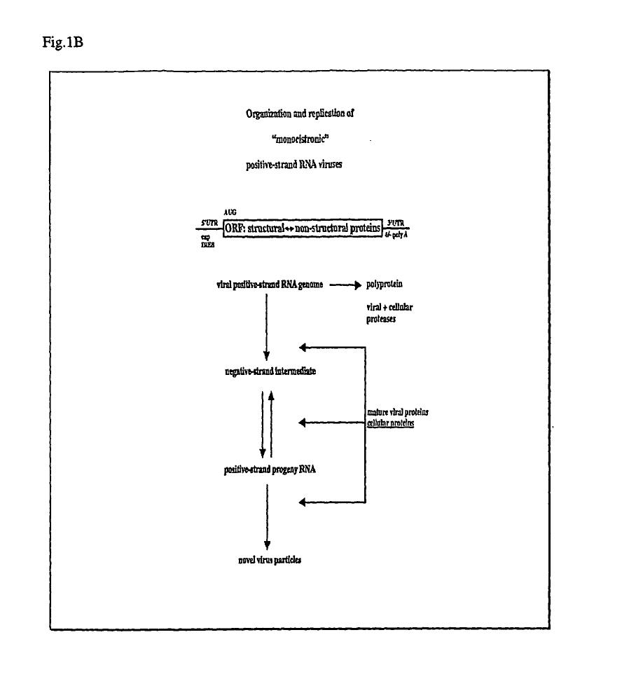

### DESCRIPTION OF THE INVENTION

The invention relates to a set of polypeptides, their production and uses, as well as variants, agonists and antagonists and their uses. In particular, in these and in other regards, the invention relates to a set of cellular polypeptides, hereinafter referred to as viral RNA binding proteins (vRbp). Preferably, vRbps include, but are not limited to vRbp130, vRbp120, vRbp110, vRbp84, vRbp67, vRbp64, and vRbp45. Evidence is presented implicating a critical involvement of these proteins in the life cycle of positive-strand RNA viruses containing type I, type II, type III and type IV IRES (internal ribosomal entry site) elements: i.e., Enterovirus, Rhinovirus, Cardiovirus, Aphtovirus, hepatitis A virus, hepatitis C virus and pestivirus. Accordingly, [vRbp130, vRbp120, vRbp110, vRbp84, vRbp67, vRbp64 and vRbp45] their potential protein interaction partners as well as their interaction-site(s) on the respective viral RNAs should be considered as targets for treatment of disease syndromes associated with infections of any of these viruses. The present invention relates to or unquestionably demonstrates that different members of the NFAT/NFAR/NF90 polypeptide family represent vRbp110, vRbp84, and vRbp64, respectively, and that the NF90 associated polypeptide NF45 represents vRbp45. vRbp120 is indicated to represent RNA helicase A (RHA). Other data implicate the proteins to regulate the coordination of translation and replication of the diverse viral genomes. Because all NFAT/NFAR/NF90 variants as well as RHA interact and/or are substrates of the dsRNA-activated protein kinase PKR, [vRbp130, vRbp120, vRbp110, vRbp84, vRbp67, vRbp64 and vRbp45] are suggested to antagonize the cellular defence mechanisms against viral infections.

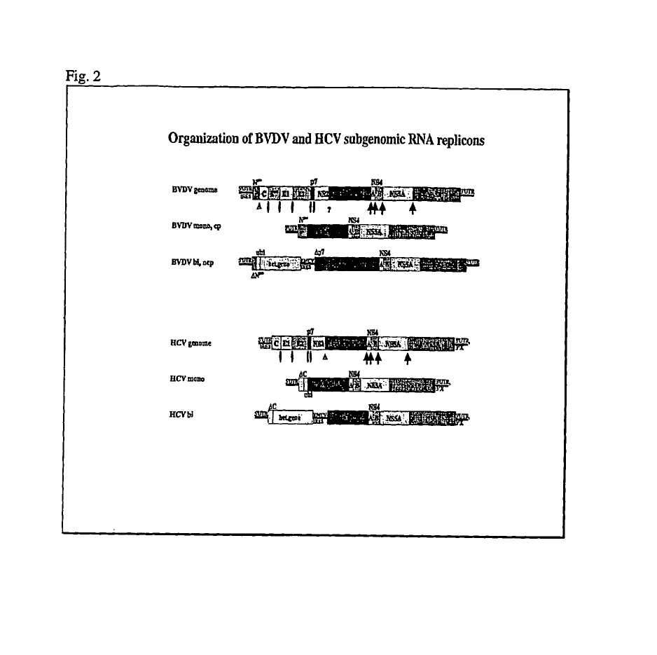

The starting point of this invention was the discovery that subgenomic BVDV RNAs that lack the coding regions of the virus structural proteins are replication competent in transfected host-cells (36). BVDV “replicon RNA” can be generated by in vitro transcription from cloned cDNA constructs; it replicates in a wide range of different host-cells (e.g. MDBK, BHK-21, human hepatocytes or HeLa cells). In the meantime, a broad spectrum of monocistronic as well as bicistronic BVDV replicons has been composed (36, 37; see also FIG. 2). Essentially, they harbor the 5′UTR and the 3′UTR of the viral genome as well as a truncated part of the viral ORF, which comprises mainly five genetic units: i.e., NS3, NS4A, NS4B, NS5A and NS5B. The N-terminus of NS3 contains a serine protease domain, which, together with the NS4A cofactor, catalyses the proteolytic cleavages of the non-structural NS3-NS5B polyprotein. The C-terminus of NS3 associates an ATPase and RNA helicase activity. NS5B represents the viral RdRp. The function(s) of NS4B and NS5A are not known (reviewed in 2). The genomic organization of the region encoding NS3 to NS5B is virtually colinear in pestiviruses and hepaciviruses. Accordingly, the finding that subgenomic RNAs encompassing the UTRs and the NS3 to NS5B coding region encode all factors and elements, which, on the part of the virus, suffice for genome amplification has recently been extended to hepatitis C virus (38; FIG. 2). In comparison with BVDV, HCV RNA replicates less efficient (ca. 10,000 versus 1000 copies of viral RNA per cell), and its replication is restricted to only one host cell-type (i.e., Huh-7 cells).

Due a number of experimental advantages with respect to full-length viral RNA, the most important of which concerns the possibility to examine RNA replication independently of events linked to RNA packaging and/or virion assembly, BVDV and HCV replicons are currently utilized to define individual components of the replication complex and to characterize their mode of activity. As a general experimental scheme, the viral RNA is mutagenized via the cDNA construct (a procedure termed as “reverse genetics”) and the effects of mutagenesis on replication are monitored using appropriate assay systems such as RNase protection or RT-PCR. Reverse genetics studies are completed by biochemical experiments such as RNA structure probing, UV-crosslinking/label transfer experiments (see below) and specific assay systems to measure the enzymatic activities of the NS3 protease, the NS3 ATPase/RNA helicase and the NS5B RdRp, respectively (39-42).

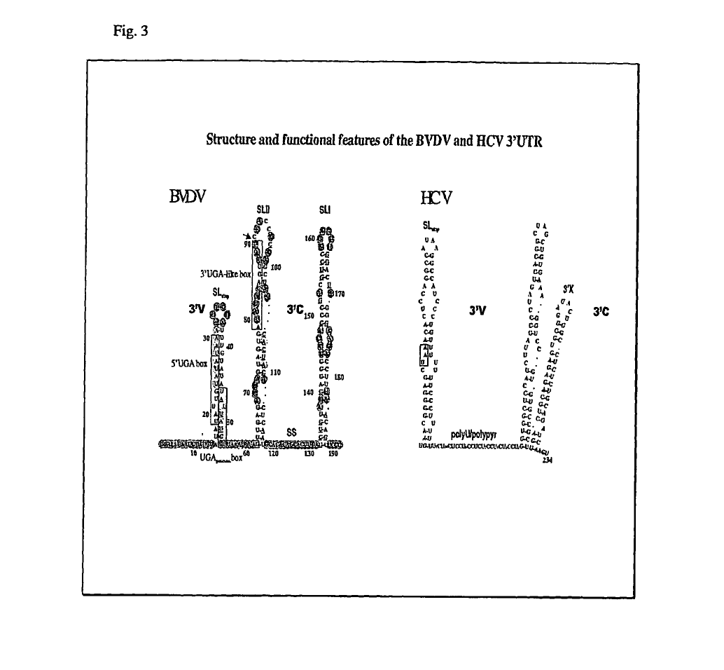

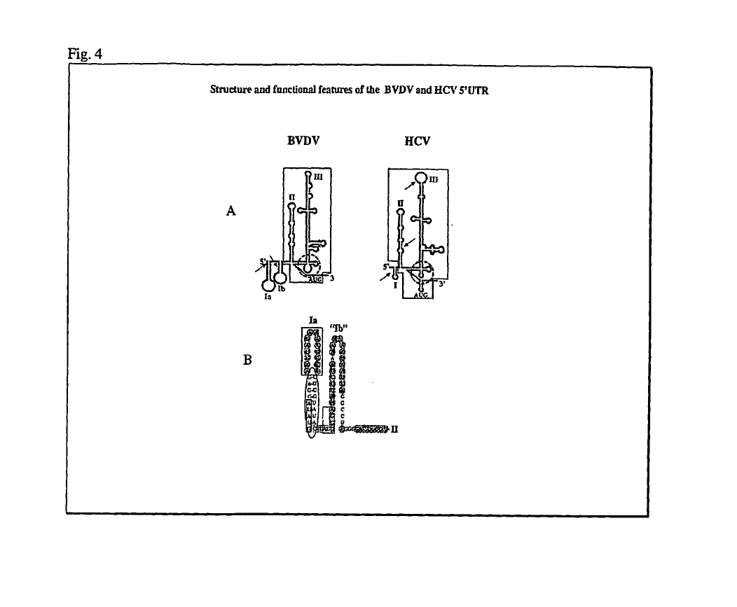

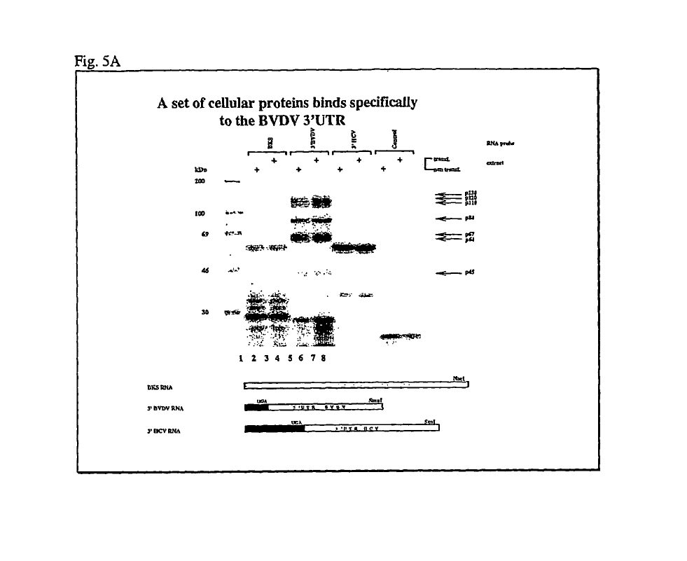

Besides serving as an experimental system to identify and to characterize the molecular determinants that control the viral RNA replication pathway, BVDV and HCV replicons proved to be useful tools to study the IRES-mediated translation process. For this purpose, in vivo translation assays were established, the most meaningful of which apply bicistronic constructs encoding a heterologous enzymatic activity such as □-glucoronidase (37; see also FIG. 2). In addition, the Applicants developed an in vitro translation assay based on cytoplasmic initiation factor fractions of authentic host cells (BHK-21 cells for BVDV; Huh-7 cells for HCV). Programmed with genuine viral RNA, the in vitro system was shown to appropriately mimic the in vivo situation of viral polyprotein synthesis and processing (39, 41, 43-45). Studies of other laboratories and Applicants of the present invention revealed the following findings related to BVDV and HCV replicon systems, which are relevant for this invention:
Taking a genetic approach to the BVDV replicon, the Applicants could show that all the mature replicon-encoded non-structural proteins NS3 to NS5B and all known virus-encoded enzymatic activities (protease, helicase, polymerase) are essentially involved in an early stage of the replication cycle. The majority of the non-structural proteins, namely NS3, NS4A, NS4B and NS5B were indicated to act in cis (e.g. in statu nascendi) during assembly of the replication complex; only one protein, NS5A, was suggested to operate in trans. In summary, these data demonstrate a close functional linkage of translation and processing of the viral proteins and their activity during replication. NS5A appears to play a particular role during viral RNA replication (39, 41).
The 5′-terminal portion of the viral ORF, which encodes the N-terminus of the autoprotease N^{pro} (pestiviruses) or the N-terminus of the capsid protein C (HCV), respectively, represents a functional entity of the IRES: i.e. this region is important for efficient translation, while it is only slightly involved in RNA replication. However, expression of an intact N^{pro} or C protein is not essential for RNA replication (36, 37, 38, 45, 46). In conclusion, on the part of the virus, only the proteins that derive from the NS3 to NS5B coding region (i.e. the fully processed NS3 to NS5B proteins and hypothetical cleavage intermediates of the NS3-NS5B polyprotein) are involved in the assembly of the pestiviral and HCV replication complex.
Structure probing and genetic approaches revealed that the highly conserved 3′-terminal portions of the BVDV and HCV 3′UTRs (termed as 3′C regions) form extensive stem-loop (SL) structures, which are essential for viral replication (42, 47-50; see FIG. 3). With BVDV, structure as well as sequence motifs of the 3′C region were shown to be part of the “negative-strand promoter” of the initial replication complex; i.e., mutations, which modified these motifs were found to block the first step of the replication cycle (42). Importantly, the BVDV 3′C region was determined to be not essential for IRES-mediated translation initiation (45).
Similar strategies identified replication signals in the 5′UTR of the BVDV and the HCV genome, respectively. With BVDV, these motifs could be exactly defined; they concern sequence elements, which are exclusively located at or near the immediate 5′-terminus of the viral RNA (43, 45, 51; see FIG. 4). With HCV, yet undefined replication signals are harbored by the 5′-terminus of the viral genome; other elements appear to be localized in the IRES domain (52 and our data 44). As a common concept, the BVDV and HCV 5′UTRs contain “bi-functional” RNA elements, which modulate the translation as well as the replication process. Along this line, the overall integrity of the BVDV hairpin la structure and of the HCV domain III were found to be important for efficient translation initiation. In addition, these motifs contain sequence elements that are essential for the replication cycle. Reminiscent of the situation with the ORF or the 3′UTR (see above), mutations, which affected the replication signals in the BVDV Ia structure were observed to inhibit already the first replication step (43, 44). This important finding suggests that not only the 3′-end but also the 5′-end of the viral genome is involved in an early step of the replication pathway (see below).
In contrast with the 3′C region of the BVDV and HCV 3′UTR, the region immediately downstream of the ORF exhibits a remarkable heterogeneity in terms of size and sequence composition. Accordingly, it was designated as the variable 3′V region of the 3′UTR (47). Sequence alignments, computer modeling of the RNA secondary structure and experimental structure probing revealed that the pestiviral as well as the HCV 3′V region harbor several conspicuous RNA features. On the one hand, these concern so-called pseudo-stop elements, i.e. stop-codon like nucleotide triplets that are organized “in frame” with the ORF (FIG. 3). On the other hand, the portion of the 3′UTR immediately downstream of the translational stop forms an extensive stem-loop structure (termed as SLₛₜₒₚ). However, the pestivirus SLₛₜₒₚ structures exhibit generally a low stability, while the analogous structure of the HCV genome appears to be rather stable (53; see FIG. 3). Another difference concerns moderately conserved A/U-rich sequence elements (termed as “UGA-boxes”; consensus sequence: 5′A/U-A-U/G/A-U-G/A-U-G/A-U/GA-A/U-G/U-A-U/G/A3′; bold-typed residues are 100% conserved among all pestivirus species), which are located in single or multiple copies downstream of the translational stop-codon of the pestivirus ORF. These elements are not present in the HCV 3′UTR. Instead, the HCV 3′V region contains a long polyU stretch and a polypyrimidine-rich region (FIG. 3). Interestingly, deletion of SLₛₜₒₚ and/or the mutagenesis of conserved nucleotides within the BVDV UGA boxes caused a lower rate of translation initiation and inhibited the replication of the altered viral RNA (53; see also below). Similarly, deletion of the HCV SLₛₜₒₚ structure was found to inhibit RNA replication. Most interestingly is the finding that despite of the aforementioned differences, the BVDV and HCV SLₛₜₒₚ structures were shown to be functionally interchangeable (see below).

Taken together, three types of functional elements of the viral RNAs can be discriminated. (i) RNA structure motifs and sequence elements at the immediate 3′-end of the viral genome, which operate exclusively as replication signals. (ii) The IRES domain, which spans a major portion of the 5′UTR as well as the 5′-terminus of the protein-coding region. Although cap-independent entry of ribosomes is generally enabled by this region (55, 56), other parts of the RNA molecule such as the BVDV Ia domain and the 3′V region of the 3′UTR of the BVDV and HCV genome (54 and our data 53) were shown to have a considerable impact on the efficiency of translation initiation. (iii) Motifs at each end of the RNA molecule (with BVDV: hairpin la at the 5′-end and the UGA box motifs in the 3′V region), which modulate translation as well as replication of the viral RNA. The bi-functional character of these elements suggests that they play a key role during regulation of translation and RNA replication.

Glossary

The following definitions are provided to facilitate understanding of certain terms used frequently hereinbefore. The following definitions are provided to facilitate understanding of certain terms used frequently hereinbefore.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Antibodies” as used herein includes polyclonal and monoclonal antibodies, chimeric, single chain, and humanized antibodies, as well as vRbp fragments.

“Polynucleotide” generally refers to any polyribonucleotide (RNA) or polydeoxribonucleotide (DNA), which may be unmodified or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, Proteins-Structure and Molecular Properties, 2nd Ed., T. E. Creighton, W. H. Freeman and Company, New York, 1993; Wold, F., Post-translational Protein Modifications: Perspectives and Prospects, 1-12, in Post-translational Covalent Modification of Proteins; B. C. Johnson, Ed., Academic Press, New York, 1983; Seifter et al, “Analysis for protein modifications and nonprotein cofactors”, Meth Enzymol, 182, 626-646, 1990, and Rattan et al., “Protein Synthesis: Post-translational Modifications and Aging”, Ann NY Acad Sci, 663, 48-62, 1992).

“Fragment” of a polypeptide sequence refers to a polypeptide sequence that is shorter than the reference sequence but that retains essentially the same biological function or activity as the reference polypeptide. “Fragment” of a polynucleotide sequence refers to a polynucleotide sequence that is shorter than the reference sequence of a vRbp.

“Variant” refers to a polynucleotide or polypeptide that differs from a reference polynucleotide or polypeptide, but retains the essential properties thereof. A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, Ile, Leu; Asp, Glu; Asn, Gln; Ser, Thr; Lys, Arg; and Phe and Tyr. A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis. Also included as variants are polypeptides having one or more post-translational modifications, for instance glycosylation, phosphorylation, methylation, ADP ribosylation and the like. Embodiments include methylation of the N-terminal amino acid, phosphorylations of serines and threonines and modification of C-terminal glycines.

“Allele” refers to one of two or more alternative forms of a gene occurring at a given locus in the genome.

“Polymorphism” refers to a variation in nucleotide sequence (and encoded polypeptide sequence, if relevant) at a given position in the genome within a population.

“Single Nucleotide Polymorphism” (SNP) refers to the occurrence of nucleotide variability at a single nucleotide position in the genome, within a population. An SNP may occur within a gene or within intergenic regions of the genome. SNPs can be assayed using Allele Specific Amplification (ASA). For the process at least 3 primers are required. A common primer is used in reverse complement to the polymorphism being assayed. This common primer can be between 50 and 1500 bps from the polymorphic base. The other two (or more) primers are identical to each other except that the final 3′base wobbles to match one of the two (or more) alleles that make up the polymorphism. Two (or more) PCR reactions are then conducted on sample DNA, each using the common primer and one of the Allele Specific Primers.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of that may encode different amino acid sequences. The term splice variant also refers to the proteins encoded by the above cDNA molecules.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

“% Identity”—For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

“Similarity” is a further, more sophisticated measure of the relationship between two polypeptide sequences. In general, “similarity” means a comparison between the amino acids of two polypeptide chains, on a residue by residue basis, taking into account not only exact correspondences between a between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J et al, Nucleic Acids Res, 12, 387-395, 1984, available from Genetics Computer Group, Madison, Wis., USA), for example the programs BESTFlT and GAP, may be used to determine the % identity between two polynucleotides and the % identity and the % similarity between two polypeptide sequences. BESTFIT uses the “local homology” algorithm of Smith and Waterman (J Mol Biol, 147,195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Needleman and Wunsch (J Mol Biol, 48, 443-453, 1970). GAP is more suited to comparing sequences that are approximately the same length and an alignment is expected over the entire length. Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25:389-3402, 1997, available from the National Center for Biotechnology Information (NCBI), Bethesda, Md., USA and accessible through the home page of the NCBI at www.ncbi.nlm.nih.gov) and FASTA (Pearson W R, Methods in Enzymology, 183, 63-99, 1990; Pearson W R and Lipman D J, Proc Nat Acad Sci USA, 85, 2444-2448, 1988, available as part of the Wisconsin Sequence Analysis Package).

Preferably, the BLOSUM62 amino acid substitution matrix (Henikoff S and Henikoff J G, Proc. Nat. Acad Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a reference polynucleotide or a polypeptide sequence, the query and the reference sequence being optimally aligned and the parameters of the program set at the default value, as hereinbefore described.

“Identity Index” is a measure of sequence relatedness which may be used to compare a candidate sequence (polynucleotide or polypeptide) and a reference sequence. Thus, for instance, a candidate polynucleotide sequence having, for example, an Identity Index of 0.95 compared to a reference polynucleotide sequence is identical to the reference sequence except that the candidate polynucleotide sequence may include on average up to five differences per each 100 nucleotides of the reference sequence. Such differences are selected from the group consisting of at least one nucleotide deletion, substitution, including transition and transversion, or insertion. These differences may occur at the 5′ or 3′ terminal positions of the reference polynucleotide sequence or anywhere between these terminal positions, interspersed either individually among the nucleotides in the reference sequence or in one or more contiguous groups within the reference sequence. In other words, to obtain a polynucleotide sequence having an Identity Index of 0.95 compared to a reference polynucleotide sequence, an average of up to 5 in every 100 of the nucleotides of the in the reference sequence may be deleted, substituted or inserted, or any combination thereof, as hereinbefore described. The same applies mutatis mutandis for other values of the Identity Index, for instance 0.96, 0.97, 0.98 and 0.99.

Similarly, for a polypeptide, a candidate polypeptide sequence having, for example, an Identity Index of 0.95 compared to a reference polypeptide sequence is identical to the reference sequence except that the polypeptide sequence may include an average of up to five differences per each 100 amino acids of the reference sequence. Such differences are selected from the group consisting of at least one amino acid deletion, substitution, including conservative and non-conservative substitution, or insertion. These differences may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a polypeptide sequence having an Identity Index of 0.95 compared to a reference polypeptide sequence, an average of up to 5 in every 100 of the amino acids in the reference sequence may be deleted, substituted or inserted, or any combination thereof, as hereinbefore described. The same applies mutatis mutandis for other values of the Identity Index, for instance 0.96, 0.97, 0.98 and 0.99.

The relationship between the number of nucleotide or amino acid differences and the Identity Index may be expressed in the following equation:
nₐ≦xₐ−(xₐ·I),
in which:
nₐ is the number of nucleotide or amino acid differences,
xₐ is the total number of nucleotides or amino acids in ROCK or ROCK, respectively,
I is the Identity Index,
· is the symbol for the multiplication operator, and
in which any non-integer product of xₐ and I is rounded down to the nearest integer prior to subtracting it from xₐ.

“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog”, and “paralog”. “Ortholog” refers to a polynucleotide or polypeptide that is the functional equivalent of the polynucleotide or polypeptide in another species. “Paralog” refers to a polynucleotideor polypeptide that within the same species which is functionally similar.

“Modulates” means in reference to an activity herein, resulting in a change in an amount, and/or quality, and/or effect of a particular response and/or activity. Both increases and/or decreases in a response and/or activity are included.

“Picornaviridae” as used herein refers to a family of single-stranded RNA-containing viruses that cause hepatitis in humans.

“Enterovirus” as used herein refers to a genus of Picornaviridae that preferentially replicate in the mammalian intestinal tract. It includes the polioviruses and Coxsackie viruses.

“Rhinovirus” as used herein refers to a genus of Picornaviridae that largely infect the upper respiratory tract. Include the common cold virus and foot and mouth disease virus.

“Cardiovirus” as used herein refers to a genus of viruses belonging to the Family Picornaviridae, isolated mostly from rodents, cause encephalitis and myocarditis.

“Hepatovirus” as used herein refers to a genus of Picornaviridae causing infectious hepatitis naturally in humans and experimentally in other primates. It is transmitted through faecal contamination of food or water.

“Aphtovirus” as used herein refers to a genus of the family picornaviridae causing foot-and-mouth disease in cloven-hoofed animals.

“Flaviviridae” as used herein refers to a family of single-stranded RNA-containing viruses that cause haemorrhagic fever in a wide range of mammals and are transmitted by mosquitos, such as West Nile Virus, and ticks.

“Flavivirus” as used herein refers to a genus of Flaviviridae, also known as group b arbovirus, containing several subgroups and species. Most are arboviruses transmitted by mosquitoes or ticks. The type species is yellow fever virus.

“Pestivirus” as used herein refers to a genus of Flaviviridae, also known as mucosal disease virus group, which is not arthropod-borne. Transmission is by direct and indirect contact, and by transplacental and congenital transmission. Species include border disease virus, bovine viral diarrhea virus (diarrhea virus, bovine viral), and hog cholera virus.

“Hepatovirus” as used herein refers to a non-A, non-B RNA virus causing post-transfusion hepatitis; it appears to be a member of the family Flaviviridae.

“Antagonist” as used herein refers to a substance that tends to nullify the action of another, as a drug that binds to a cell receptor without eliciting a biological response.

“Agonist” as used herein refers to a substance that has affinity for and stimulates physiologic activity at cell receptors normally stimulated by naturally occurring substances, thus triggering a biochemical response.

“Fusion protein” refers to a protein encoded by two, often unrelated, fused genes or fragments thereof In one example, employing a fusion protein is advantageous for use in therapy and diagnosis resulting in, for example, improved pharmacokinetic properties. On the other hand, for some uses it would be desirable to be able to delete part of a protein.

“vRbp130” as used herein refers to a post-translational modification of RNA helicase A.

“vRbp120” as used herein refers to a complex with NF90/NFAR1 and NF45 (RNA helicase A or RHA).

“vRbp110” as used herein refers to an alternatively spliced form of NFARI (NFARII).

“vRbp84” as used herein refers to a C-terminally modified NF90 (NFARI).

“vRbp67” as used herein refers to a 64 kDa subunit of cleavage stimulatory factor (CSTF) involved in polyadenylation of mRNAs, which however, does not bind specifically to viral RNAs.

“vRbp64” as used herein refers to an alternatively spliced form of NFARI and NFARII.

“vRbp45” as used herein refers to a complex with NF90/NFAR1 and RNA helicase A (NF45).

“Cross-Talk” as used herein refers to extensive interactions between the viral termini (3′ and 5′UTR), or interactions between the structural elements within the 3′ntr and the stop codon in NS5B are likely to be critical in regulating translation termination, translational frameshifting and the coordinated balance of replication and translation on the positive strand RNA. As HCV is an RNA virus, the viral RNA forms highly ordered secondary and tertiary conformations. Many of these conformations have been determined by biophysical probing, such as that for the 5′ntr. It is equally likely, that the ordered stem-loop structures of the RNA are critical to control translation and replication. Circularization of the viral genome may occur directly via the UTRs or facilitated by the UTR along with said cellular proteins bound to the UTR. Additionally, multiple contacts of the UTR RNA, or UTR RNA with said cellular proteins bound, may interact with other regions of the viral genome.

In addition, the present invention relates to methods of interfering with the translational regulation and replication of HCV RNA could occur by providing excess amounts of 3′UTR RNA, or 3′UTR RNA elements which are required for interacting with the said cellular proteins. In effect, providing an exogenous source of viral RNA capable of binding the said cellular proteins should effectively serve as a sink, to titrate out the ‘activity’ of these cellular proteins. If they were sufficiently removed from the test system, viral replication should be substantially reduced. Since these proteins may be directly required for viral replication, and their availability to interact with the authentic viral genome becomes limited upon effective binding to the RNA decoy sink, viral replication should be decreased. Additionally, removal of these cellular proteins from binding the authentic viral genome, may result in the loss of coordinated regulation between translation and replication. An decrease in accurate termination of translation would be expected to be a direct outcome of the loss of these cellular proteins binding to the authentic UTR of the viral genomes. Upon increased translation beyond the authentic stop codon in NS5B, steric hindrance or competition between the ribosomes (for translation) and the initiation of viral RNA synthesis by the replicase complex binding to the 3′UTR, should result in a direct decrease in viral replication. Also, systems utilizing peptide-nucleic acid conjugates may represent a more attractive approach to creating a functional sink with nucleic acids, and with such sinks as having improved DMPK properties over conventional nucleic acids.

“Reticulocyte lysate translation assay” as used herein refers to methods for modulating a fraction of said cellular proteins within translation extract (luciferase RNA), should result in modulation of luciferase activity and therefore translation. In addition, the assay can also (i) measure impact on PKR, (ii) look at UTRs or mutant UTRs (containing mutations within binding sites for said cellular proteins) to modulate translation, and (iii) monitor compound interference.

“Cell-based translation frameshift assay” as used herein refers to methods for assays that identify compounds that would be predicted to enhance translational frameshifting, and/or decrease translation termination at authentic stop codon. Compounds capable of doing this would be expected to result in ribosomes moving 3′ from the stop codon, and represent a steric hindrance for replicase protein binding. The assay can (i) monitor by ELISA for small peptide generated by this frameshift, (ii) could use a BRET assay to monitor the interaction of said cellular proteins from 5′UTR with said cellular proteins binding 3′UTR, and (iii) can be a measure of genome circularization.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### EXAMPLES

The invention is further illustrated by way of the following examples which are intended to elucidate the invention. These examples are not intended, nor are they to be construed, as limiting the scope of the invention. It will be clear that the invention may be practiced otherwise that as particularly described herein. Numerous modifications and variations of the present invention are possible in view of the teachings herein and, therefore, are within the scope of the invention. The examples below are carried out using standard techniques, which are well known and routine to those of skill in the art, except where otherwise described in detail.

### Example 1

### A Set of Ubiquitous Cellular Proteins Binds to the 5′ and 3′UTR of Pestiviral RNA and is Critically Involved in Translation and RNA Replication

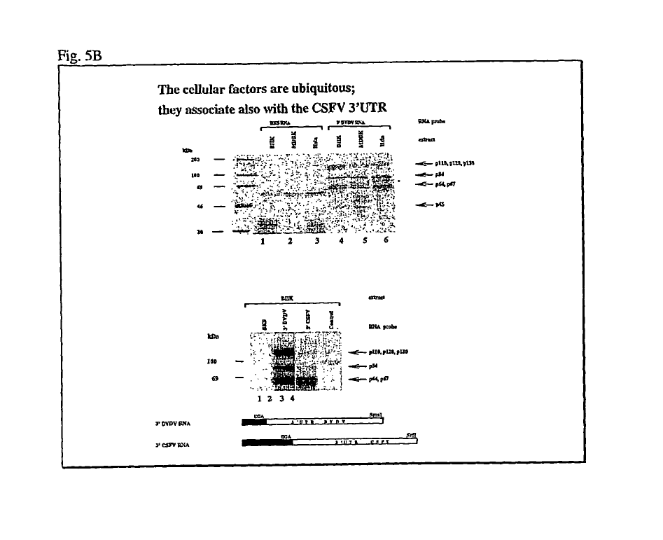

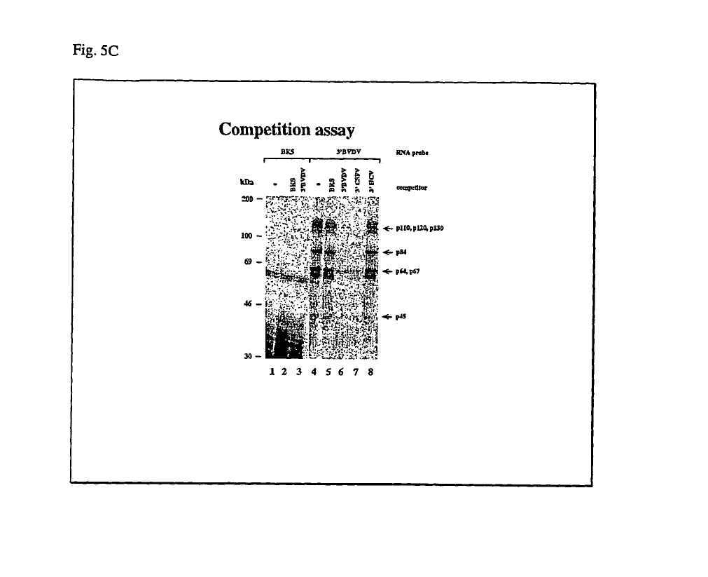

The new invention concerns a set of RNA-binding proteins (termed as vRbp 130, vRbp120, vRbp 110, vRbp84, vRbp67, vRbp64 and vRbp45), which were originally identified by UV crosslinking/label transfer approaches to bind to the UGA-box elements of the BVDV 3′V region (53). Competition experiments demonstrated that binding of vRbp130, vRbp120, vRbp110, vRbp84, vRbp64 and vRbp45 to the viral RNA is highly specific. vRbp67 was determined to bind in a non-specific manner (53; see FIG. 5). The vRbp “host-factors” are ubiquitous in all cell-types that support BVDV replication (FIG. 5), and they can be fractionated from a ribosomal salt wash (53). The latter result suggested that several of these proteins represent non-canonical components of the cellular translation apparatus (see below). While the specific binding factors vRbp130, vRbp120, vRbp110, vRbp84, vRbp64 and vRbp45 were suggested or evidently shown to represent different members of dsRNA binding proteins (for details, see below), the non-specific RNA-binding protein vRbp67 was demonstrated to correspond to the 64 kDa subunit of cleavage stimulatory factor (CSTF) (reviewed in reference 57).

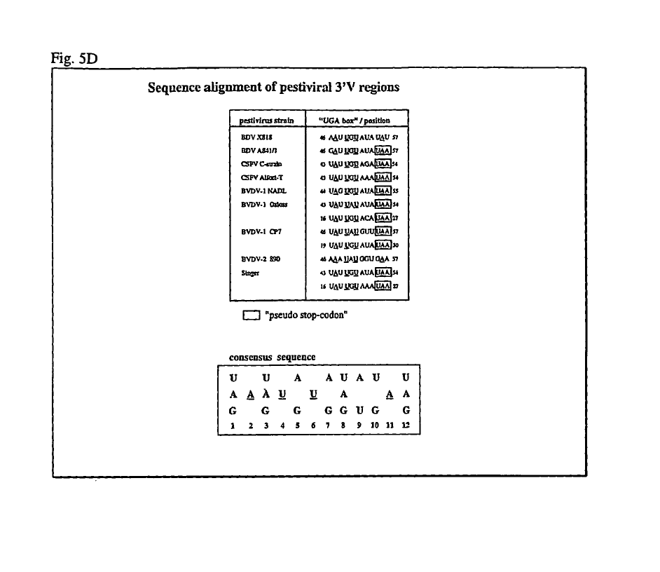

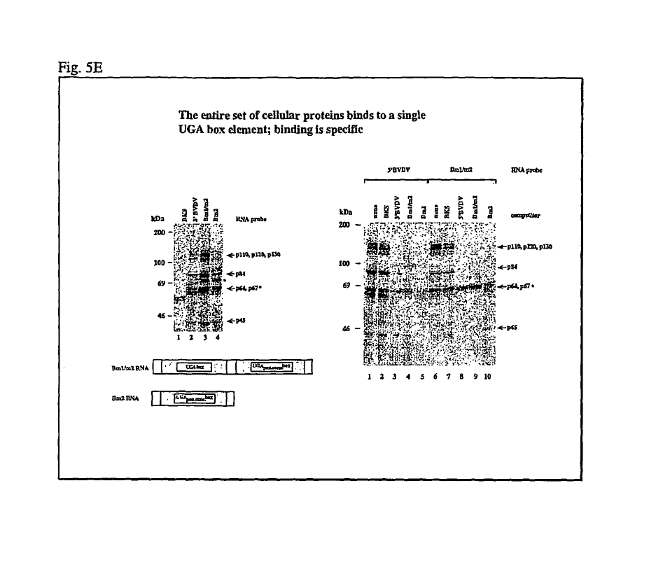

Importantly, binding of the vRbps to the UGA elements correlated strictly with the ability of BVDV replicon RNA to amplify within the host-cell. Moreover, the interaction of these cellular factors with the 3′V region was indicated to be essential for the clearance of translating ribosomes from the viral RNA. Thus, mutant BVDV RNAs containing deletion and/or point mutations, which changed the sequence of the UGA box and pseudo-stop elements (the latter, which are mostly part of the UGA box consensus sequence) and which modified the folding of SLₛₜₒₚ and SLII of the 3′V region, were found to associate the vRbps to a significantly lesser extent (FIG. 6). As mentioned above, the efficiency of translation initiation of these mutant RNAs was found to be reduced, and, most strikingly, proper termination of translation was observed to be impaired, i.e. a significant read-through of the translational stop-codon of the ORF by ribosomes could be detected (FIG. 6). Consistent with the idea that translation and replication are mutually exclusive events (see above), and that incomplete translation termination should interfere with the assembly of the functional replication complex, viral RNA derivatives encoding thus modified 3′V regions turned out to be replication deficient (FIG. 6). As explained further below, analogous results were obtained with HCV RNA (44, 53).

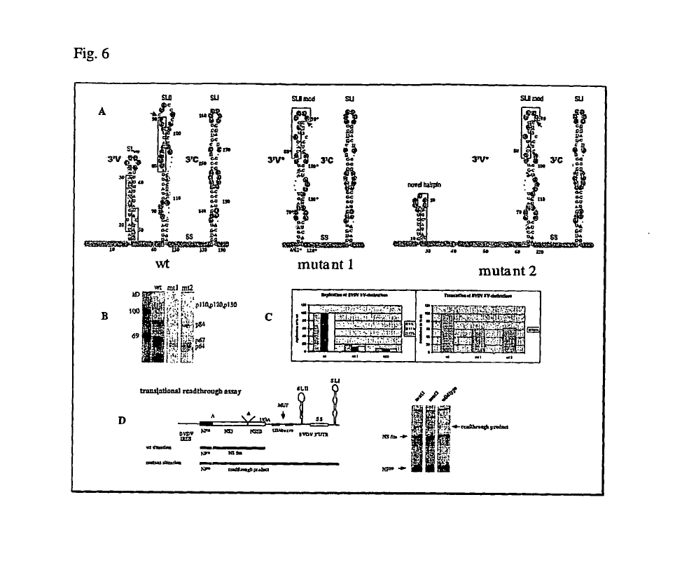

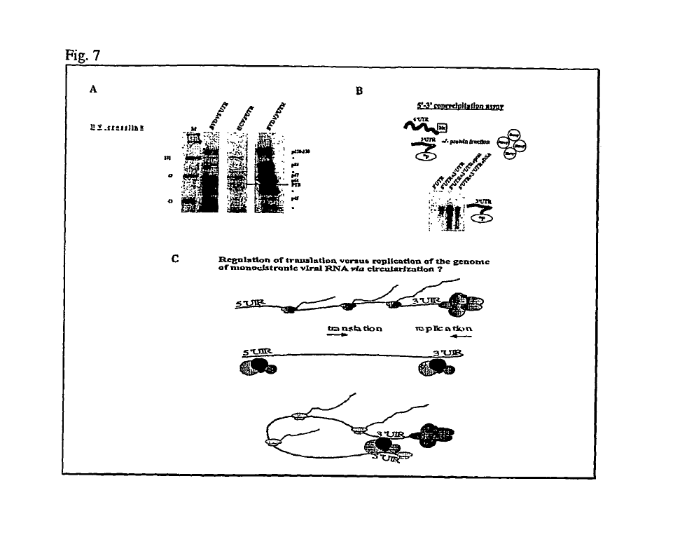

A further series of crosslinking and competition experiments demonstrated that the identical range of factors (vRbp 130, vRbp120, vRbp110, vRbp84, vRbp64 and vRbp45) bind also specifically to the BVDV 5′UTR (FIG. 7). Importantly, binding of the vRbp proteins to the 3′V region could be competed with transcripts consisting of the 5′UTR and vice versa (53). The RNA-protein interaction site(s) within the BVDV 5′UTR (note that the 5′UTR does not contain UGA box like sequence elements) has not yet been defined. However, initial indications came from experiments showing that hairpin Ia mutations, which inhibited translation and/or RNA replication, respectively [symbol] , reduced the capability of the BVDV 5′UTR to associate the vRbps (53). Since hairpin Ia per se does not bind the proteins (53), the RNA-protein interaction domain is suggested to represent a complex RNA motif, which involves also hairpin Ia (see also below).

As an important result which suggests a host-factor mediated 5′-3′ cross-talk of the viral RNA, fractions containing vRbp84 and vRbp45 were found to precipitate radioactively labeled transcripts covering the 3′UTR via biotinylated transcripts which encompass the 5′UTR (FIG. 7). These experiments are currently repeated with the purified NF90NFAR-1 and NF45 proteins.

In summary, the presented data provide evidence for the formation of a specific viral/cellular RNP complex critically involved in translation and RNA replication or the coordinated regulation of translation and replication of BVDV RNA. (i) Association of the vRbps with the viral RNA involves the aforementioned “bi-functional” RNA motifs: i.e., the hairpin Ia structure at the 5′-end and the UGA box elements at the 3′-end of the RNA. (ii) Inhibition of binding of the vRbps to the 5′ or 3′-end of the viral RNA strictly correlates with inhibition of translation and/or replication of the viral RNA. (iii) The modification of UGA box elements in the 3′UTR cause a less efficient termination of translation. Accordingly, the replication deficiency of UGA box mutants may be explained by a disturbed coordination of translation versus replication, or, in other words, by an interference of the translation with the replication machinery. (iv). As strongly indicated by the coprecipitation experiments, simultaneous binding of the vRbps to the 5′ as well as to the 3′-end may bring about a physical and functional link between both ends of the viral RNA and may thus enable feed-back regulation between the translation and replication machinery (FIG. 7). Along this line, it is possible that the RNP complex and associating viral protein(s) (e.g. NS5A) contribute to the displacement of ribosomes from the RNA. Alternatively, it is conceivable that the state of the assembling replication complex at the 3′-end of the viral RNA modulates translation initiation via 3′-5′ cross talk (53).

### Example 2

### The Same Set of Cellular Proteins Associates with the UTRs of Different Types of Picornaviruses and Hepatitis C Virus

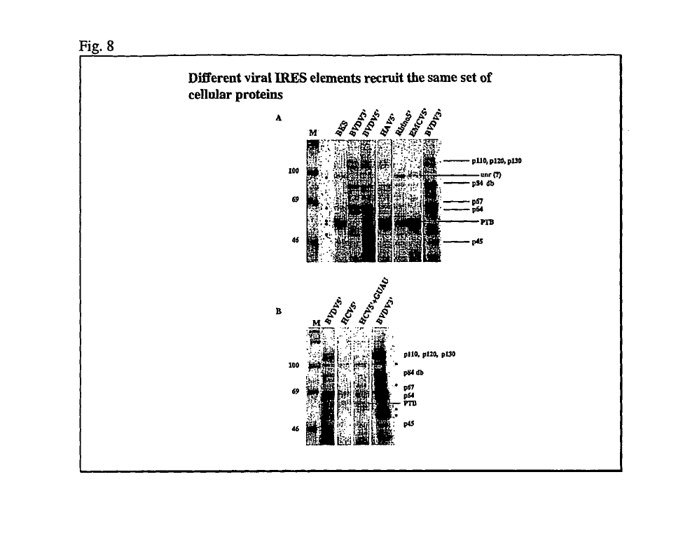

Association of the entire set of Rbps (vRbp130, vRbp120, vRbp110, vRbp84, vRbp64 and vRbp45) was also detected with the 5′UTR and 3′UTR of other pestiviruses such as CSFV (53). Moreover, the vRbps were determined to bind also to the UTRs of several other RNA viruses (FIG. 8). (i) Although the cross-linking signals were weak, binding of these factors to the 5′UTR of HCV was clearly detectable. No label-transfer occurred with different transcripts of the HCV 3′UTR (see below). (ii) Intriguingly, an identical label transfer pattern was observed during cross-linking experiments which applied the 5′UTR or the 3′UTR of HAV (hepatitis A virus) strain HM175 (FIG. 8; the 3′data were already published by Kusov et al., 58). (iii) Binding of the vRbps was also found with the 5′UTR of Rhinovirus type 14 and (iv) the 5′UTR of EMCV (encephalomyocarditis virus) (FIG. 8). The 3′UTRs of the latter viruses have not yet been tested. Hence, association of vRbp130, vRbp120, vRbp110, vRbp84, vRbp64, and vRbp45 was observed with the 5′UTR of viruses harboring a type I (Entero-/Rhinoviruses), type II (Cardio-/Aphtoviruses), type III (Hepatitis A virus) or type IV (hepatitis C virus/pestiviruses) IRES element. As with the BVDV and CSFV 5′ and 3′UTR, the specificity of the RNA-protein interactions was confirmed by cross-competition experiments: for example, the association of the proteins to the HCV 5′UTR could be chased by RNA transcripts comprising the HAV 5′UTR etc. In contrast, non-related RNAs, such as t-RNA or diverse mRNA transcripts did not compete the binding of the proteins to the viral RNAs (53).

As shown in FIG. 8, the amounts of transferred label differed significantly between the various test RNAs. Considering that the supposed protein interaction site(s) (see below) of each of the different 5′UTRs comprise a variant number of labeled nucleotides, the data are difficult to interpret in terms of the efficiency of a certain RNA/protein interaction. Once the identified vRbps (see below) become available in purified form, more meaningful techniques can be applied to confirm the efficiency of the interaction of these factors to the different UTRs as well as to elements (such as the HCV 3′UTR), which yielded a negative result during crosslinking experiments.

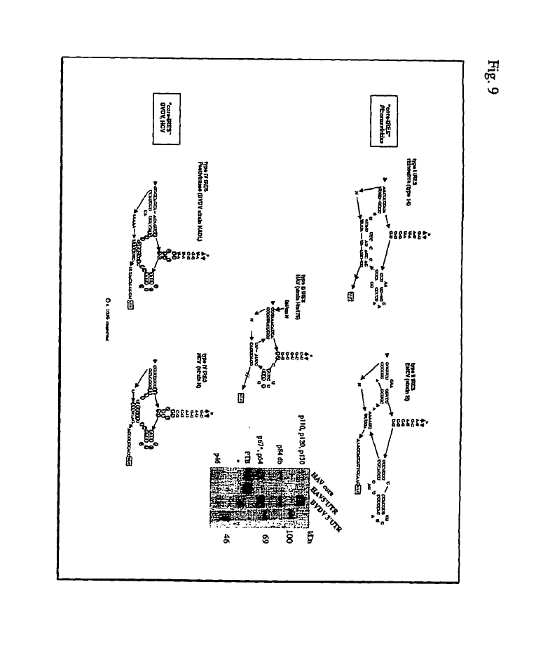

As already mentioned, neither the 5′UTRs of the diverse members of the Picornaviridae family nor the 5′UTRs of hepatitis C virus or pestiviruses contain UGA box-like sequence elements. However, despite limited sequence identity, the structural and functional organization is highly shared between IRESes of the same type (e.g. between HCV and pestiviruses, see also FIG. 4). Moreover, computer derived RNA folding and phylogenetic comparative analyses suggested a common “IRES core-domain” for the different picornaviruses as well as for the divergent hepatitis C virus and pestiviruses (59). Tests whether this structure motif, which involves approximately 100 nucleotides near the translation initation codon, and which covers the 40S interaction domain (see above), may represent the common binding site of vRbp130, vRbp120, vRbp110, vRbp84, vRbp64 and vRbp45 within the picornavirus, HCV and pestivirus IRES are underway. Initial indications that the core-IRES may represent a part of the protein binding site came from an RNase H digestion approach, which allowed the purification of the correctly folded 3′ 150 nucleotides of the 5′UTR of HAV. As shown by UV crosslinking, this region, which corresponds almost exactly to the proposed IRES core-domain, assembles indeed the entire set of vRbps (FIG. 9). Considering the core-IRES as a major vRbp binding site, the fact that with the BVDV system formation of the 5′-terminal hairpin Ia motif was found to be important for efficient interaction of the vRbps with the 5′UTR may be interpreted in two ways. (i) Formation of hairpin Ia may have a cooperative effect on the folding of the IRES core-domain and/or (ii) elements of hairpin Ia are in contact with parts of the core IRES (see also below). Taken together, these data suggest that the set of vRbp proteins associate with a complex, common RNA motif harbored by the 5′UTRs of the different virus species.

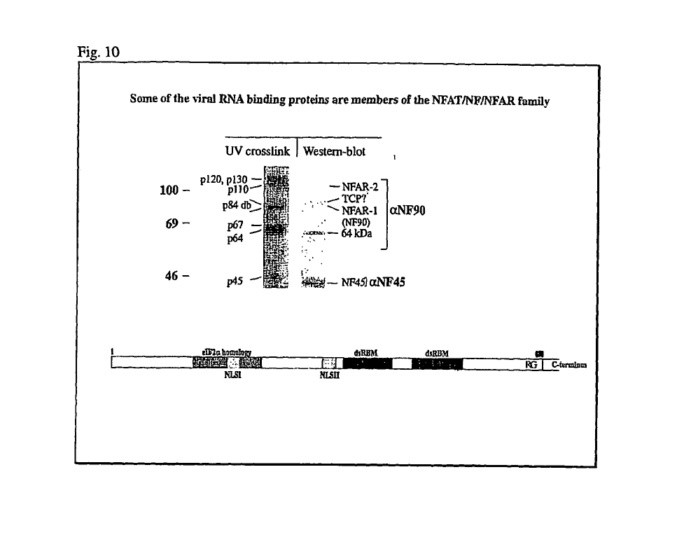

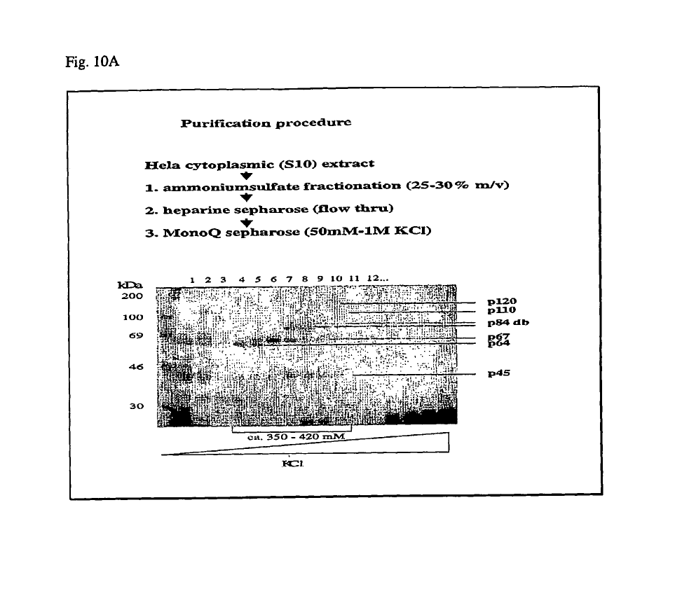

By chromatographic methods, the Applicants purified vRbp84 from cytoplasmic fractions of HeLa cells and determined its identity by mass-spectroscopy. The cellular factor identified herein, namely a member of the NF90 family (see below), was distinct from those reported by other laboratories as to interact with the genomes of picornaviruses, HCV and pestiviruses, respectively (see above). The fact that NF90 (or a close relative of this protein, see below) corresponds to the originally detected vRbp84 was verified by three types of experimental procedures: (i) By comparison of the gel retardation factor (RF) of the immunostained and the crosslinked/labeled protein on SDS-PAGE (FIG. 10). (ii) Via RNA coprecipitation experiments with the in vitro translated [³⁵S]-labeled NF90; i.e. coprecipitation of the protein could be exclusively detected with a specific RNA probe but not with an unrelated RNA (FIG. 10). (iii) By RMSA (RNA mobility shift assays) and “super-shifts” with □NF90 antibodies and different viral 5′UTRs (FIG. 10).

NF90/NFAR-1 is a double-stranded RNA binding protein, which has been originally characterized as a NFAT (nuclear factor of activated T cells)-binding component of the antigen receptor response element (ARRE) from the interleukin 2 promoter (60). Subsequently, it was designated as NF90 and NFAR-1, respectively (61, 62). The protein, which is present in the nucleus as well as in the cytoplasm of the cell, harbours a bipartite nuclear localization domain (NLS) and two dsRBMs (double-strand RNA binding motifs; reviewed in reference 63; see also FIG. 10). The coding gene is the so-called interleukin enhancer binding factor 3 gene (ILF3), which has been mapped to chromosome 19 in humans and to chromosome 9 in mice. The human gene spans 38 kb and is divided into 21 exons. Different reports indicate that a series of isoforms are expressed due to alternative splicing of the same mRNA. The protein isoforms diverge only at the carboxiterminal region of the proteins (64). Besides NF90 and NFAR-1 which differ for other reasons (see below) by 109 AA at the C-terminus, two isoforms were so far characterized. These are the TCP (“translational control protein”; 65), which, with respect to NFAR-1, differs by 15 AA at the C-terminus and contains 62 additional AA residues, and the NFAR-2, which differs by 15 AA at the C-terminus and contains 192 additional AA residues (62). Interestingly, different members of this protein-family are identified by autoantibodies of patients and mice with systemic autoimmune diseases (66). NF90/NFAR-1 was shown to bind to and to be a substrate of PKR (67). In accord with this finding, NFAR-1 and NFAR-2, which are suggested to be involved in gene-expression processes (62, 68), share a striking homology with eIF2□ (62).

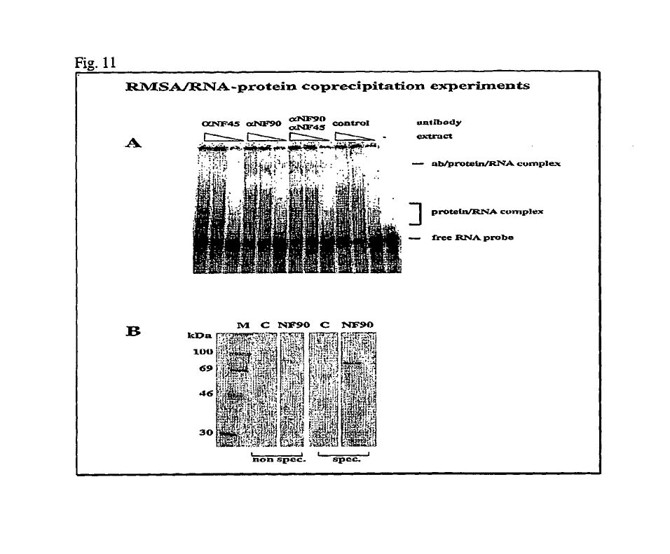

In the course of the cloning procedure, the Applicants found that the only difference between the cDNA of NF90 and NFAR-1 concerns a two base-pair frame-shift in the NF90 cDNA clone of Kao et al., which consequently leads to the expression of a different C-terminus of NF90. In other words, NF90 and NFAR-1 are not alternatively spliced forms of the same mRNA but represent most probably the same protein. In conclusion, the so-called NF90 protein family consists of three known members: NF90/NFAR-1 (calculated molecular weight, ca. 78 kDa), TCP (differs by ca. 7 kDa with respect to NFAR-1—accordingly, it has a calculated molecular weight of ca. 85 kDa) and NFAR-2 (calculated molecular weight, ca. 99 kDa). The N-terminal 690 AA residues are identical in all three family members (FIG. 10). Accordingly western-blots performed with an □NF90 antiserum (61) on total cytoplasmic proteins of HeLa cells stained a set of protein bands migrating at molecular weights of about 84, 90 and 110 kDa on SDS PAGE (FIG. 11), which were suggested to correspond to NF90/NFAR-1, TCP and NFAR-2, respectively. Moreover, the □NF90 antiserum clearly stained a protein with a molecular weight of 64 kDa, which was accordingly suggested to represent a yet unknown, additional isoform of the NF90 family (FIG. 11).

Strikingly, the overall pattern and the RF values of the proteins that are stained by the □NF90 antiserum are virtually congruent with the pattern and RF values of the vRbps labelled during UV cross-linking/label transfer experiments with the different viral RNA probes (see FIG. 11 and above). In view of these data, it is reasonable to suggest that vRbp110 corresponds to NFAR-2 and that vRbp64 represents the aforementioned 64 kDa NF90 isoform. vRbp84, which with HeLa extracts generally separates as a double band on SDS-PAGE (see FIG. 5), should thus correspond to NF90/NFAR-1 and TCP, respectively.

In the course of the purification procedure, vRbp84 was found to co-fractionate with vRbp45 (53). Considering vRbp110, vRbp84 and vRbp64 as members of the NF90 family, it was a natural suspicion that vRbp45 represents the so-called NF45 protein. NF45 was previously shown to form a stable complex with NF90 (61) and to modulate the function of NF90 (67). NF45 has a distant homology to the prokaryotic transcription factor □-54; like NF90, it is a substrate of PKR phosphorylation (67). Western-blots and RMSA with □NF45 antiserum (see FIGS. 10 and 11) as well as RNA-protein coprecipitation experiments confirmed that vRbp45 represents indeed NF45.

Observations of other laboratories make it very likely that vRbp120 corresponds to RNA helicase A (RHA), which represents a further dsRNA binding protein. RHA is suggested to play a pivotal role in the regulation of transcription of the cell (reviewed in reference 63). This assumption is based on experiments with adenoviral RNAs, which associate NF90, NF45 and RNA helicase A. Coprecipitation experiments indicated that RNA helicase A (MW ca. 120-130 kDa) is tightly associated with NF90 and NF45 (69). Thus, with the exception of vRbp130 (which is suspected to represent a modification of RNA helicase A), the Applicants have direct and indirect evidence of the identity of the entire set of vRbps. Experiments applying the purified proteins to unambiguously confirm the identity of vRbp130, vRbp120 as well as that of vRbp110 and vRbp64 are in progress.

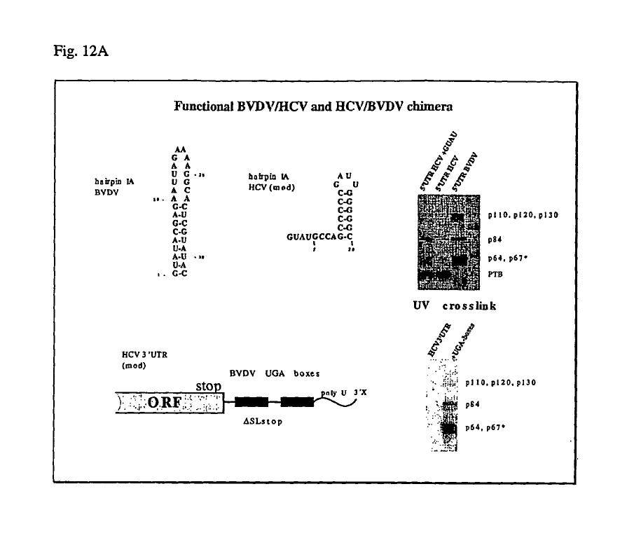

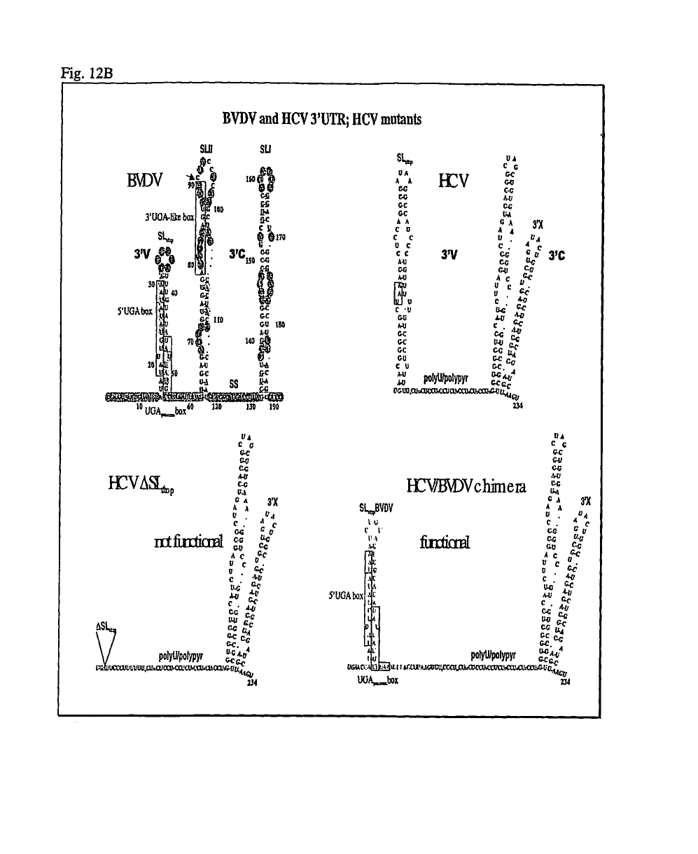

The striking similarities of pestiviruses and HCV concerning in particular the organization of the 5′UTR and of the NS3 to NS5B coding region, suggest a similar mode of translation initiation/termination, RNA replication and the coordination of both processes (see FIG. 6). Apart from the crosslinking/label transfer data shown in FIG. 8, the Applicants accumulated further evidences indicating an important functional role of the here-described vRbp proteins in the life-cycle of HCV. (i) The interaction of the vRbps with the HCV IRES was observed to be significantly stimulated if the HCV 5′UTR acquired four nucleotides “GUAU” (corresponding to the essential part of the BVDV hairpin Ia motif, see FIG. 4) at the 5′-terminus (FIG. 12). In agreement with this observation, chimeric BVDV RNA (BVDV viral genome with the 5′BVDV UTR replaced by the HCV 5′UTR) was shown to be unable to replicate; however, upon fusion of GUAU to the 5′-nd of the HCV 5′UTR, replication of this chimera was restored (37). Although these data are difficult to interpret, they support the above idea of a hairpin Ia-IRES core-domain interaction as well as of a vRbp-mediated crosstalking of the 5′ and 3′-end of the viral RNA. (ii) Despite of the aforementioned differences between the 3′V regions of the BVDV and HCV 3′UTRs (see FIG. 3), genetic data strongly suggest an analogous functional role of the BVDV and the HCV 3′V portion. Thus, deletion of the HCV SLₛₜₒₚ structure (which includes also the pseudo-stop element; see FIG. 3) yielded an RNA derivative, which is replication deficient (FIG. 6). Intriguingly, a HCV/BVDV chimera where the HCV SLₛₜₒₚ was substituted by the BVDV SLₛₜₒₚ (the latter, which associates the entire set of vRbps through the contained UGA box elements), turned out to be replication competent (FIG. 12). Studies examining whether the replication deficiency of the HCV □SLₛₜₒₚ mutant is caused by the read-through of ribosomes of the translational stop-codon are in progress.

In sum, these data implicate the here-characterized set of vRbps directly in HCV translation and replication. The detailed knowledge on the function of these newly identified vRbp factors is hence expected to considerably help to explore particular features of the RNA replication pathway, host range and pathogenesis (e.g. the reasons for chronic infections) of this insidious pathogen. To examine whether the host range of HCV (see below) may be defined by the efficiency of RNP formation, the functional, chimeric HCV/BVDV replicons are currently tested in terms of their replication capability in cells other than human hepatoma cells.

Methods capable of i) modulating the binding of cellular proteins to their supposed common binding site on viral RNA, the entire IRES core-domain, or yet undefined elements herein, or ii) modulating the biological activity of agonists and antagonists of yet unknown identity, for example viral proteins, would be valuable to prevent or treat diseases induced by divergent viruses.

The present invention relates to a specific interaction between a set of cellular proteins and the untranslated regions of a broad range of different viral RNAs. In particular, interactions involving all known types of viral IRES elements. The specificity of the formation of the viral RNA/cellular RNP complex was demonstrated by cross-linking and competition data as well as by coprecipitation experiments and RNA mobility shift assays, which were performed with individually expressed proteins and/or specific antisera, respectively (see FIG. 10 and FIG. 11). The identification of vRbp84 and vRbp45 as NF90/NFAR-1 and NF45 by purification, microsequencing and/or biochemical and immunological procedures enabled conclusions on the identity of the other vRbps. Thus, vRbp 64 and vRbp 110 were indicated to correspond to related isoforms of NF90/NFAR-1, while vRbp120 was suggested to represent RNA helicase A.

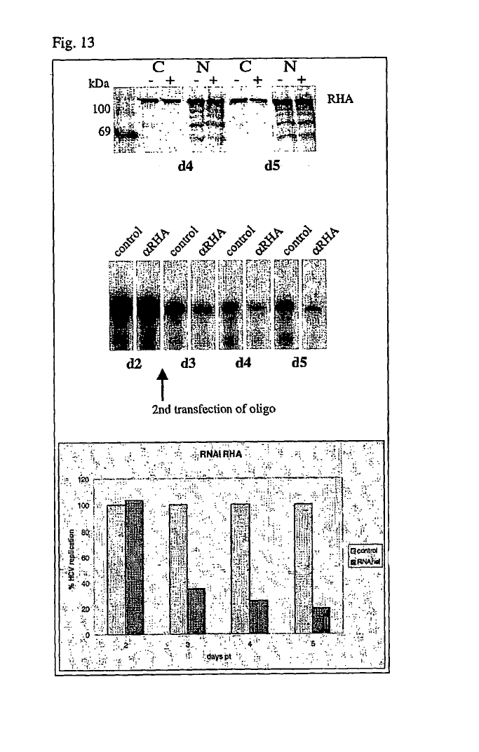

The Applicants show the importance of vRbp120 for BVDV and HCV by RNAi approaches (see FIG. 13). These approaches indicated that HCV viral replication is inhibited in vRbp120 knockouts. As indicated by the data that derived from the BVDV and HCV systems, the function(s) of the cellular vRbps appear to be critically associated with translation and replication of the viral RNA or the regulation of both processes. The fact that NF90/NFAR-1 and its relatives as well as NF45 and RHA are phosphorylated by PKR and that NF90/NFAR-1 and NF45 bind to PKR, suggests that the formation of the viral/cellular RNP may have the task to modulate the function of PKR by inhibiting its antiviral activity (see reference 67). Using antisera against the vRbp120, the Applicants were able to perform RMSA and colocalization studies via IF. Moreover, by IF, the Applicants determined that the NFs and vRbp120 (RHA) are translocated from the nucleus to the cell's cytoplasm in transfected cells.

The efficiency of the formation of the viral/cellular RNP complex may thus represent a molecular determinant of the host-range of the viral RNA. Hence, the limited host-range of HCV with respect to pestiviruses may be a consequence of the low capability of the HCV RNA to assemble the vRbps.

Taken together, the present invention suggests a universal role of dsRNA binding proteins, particularly several members of the NF90 family as well as of NF45 and RNA helicase A, in the life cycle of Picornaviruses and Flaviviruses. The development of strategies capable to inhibit either binding of the vRbps to their supposed common binding site on the viral RNA, the entire IRES core-domain or yet undefined elements herein, or the biological activity of agonists and antagonists of yet unknown identity, for example viral proteins, would be valuable to treat diseases induced by these divergent viruses.

All documents cited herein and patent applications to which priority is claimed are incorporated by reference herein in their entirety. This invention is not to be limited in scope by the specific embodiments described herein. Indeed, various modifications of the invention in addition to those described herein will become apparent to those skilled in the art from the foregoing description. Such modifications are intended to fall within the scope of the appended claims. The disclosures of the patents, patent applications and publications cited herein are incorporated by reference in their entireties.

### REFERENCES REFERRED TO HEREIN

1 Racaniello, V. R. 2001. In: Virology, 4th edition edited by B. N. Fields, Lippincott-Raven Philadelphia, N.Y.: 685-722.
2 Lindenbach, B. D., and Rice, C. M. 2001. In: Virology, 4th edition edited by B. N. Fields, Lippincott-Raven Philadelphia, N.Y.: 991-1042.
3 Pelletier, J. and Sonenberg, N. 1988. Nature 334: 320-325.
4 Jang, S. K et al. 1992. J. Virol. 62: 2636-2643.
5 Bandyopadhyay, P. K. et al. 1992. J. Virol. 66: 6249-6256.
6 Borman, A., and Jackson, R. J. 1992. Virology 188: 685-696.
7 Tsukiyama-Kohara, K. et al. 1992. J. Virol. 66: 1476-1483.
8 Poole, T. L. et al. 1995. Virology 206: 750-754.
9 Reynolds, J. E. et al. 1995. EMBO J. 14: 6010-6020.
10 Chen, C., and Sarnow, P. 1995. Science 268: 415-417.
11 Hentze, M. W. 1997. Science 275: 500-501.
12 Anthony, D. D., and Merrick, W. C. 1991. J. Biol. Chem. 266. 10218-10226.
13 Pause, A. et al. 1994. EMBO J. 13: 1205-1215.
14 Pestova, T. V. et al. 1998. Genes Dev. 12: 67-83.
15 Kieft, J. S. et al. 2001. RNA 7: 194-206.
16 Vagner, S. et al. 2001. EMBO reports: 893-898.
17 Sachs, A. B. et al. 1997. Cell 89: 831-838.
18 Lai, M. M. C. 1998. Virology 244: 1-12.
19 Hahm, B. et al. 1998. J. Virol. 72: 8782-8788.
20 Spanberg, K. and Schwartz, S. 2000. J. Gen. Virol. 80: 1371-1376.
21 Wood J. et al. 2001. J. Virol. 75:1348-1358.
22 Waggoner S., and Sarnow, P. 1998. J. Virol. 72:6699-6709.
23 Blackwell, J. L., and Brinton, M. A. 1997. J. Virol. 72: 6433-6444.
24 Spanberg, K. et al. 2000. Virology 274: 378-390.
25 Gontarek, R. R. et. al. 1999. Nucl. Acids Res. 27: 1457-1463.
26 Agol, V. I. et al. 1999. Virus Research 62: 129-147.
27 Wells, S. E. et al. 1998. Mol. Cell 2: 135-140.
28 Garmarnik, A., and Andino, R. 1998. Genes Dev. 12: 2293-2304.
29 Simoes, E. A., and Sarnow, P. 1991. J. Virol. 62: 2291-2299.
30 Herold, J. personal communication.
31 Herold J., and Andino, R. 2001. Mol. Cell 7: 581-591.
32 Khromykh, A. A. et al. 2001. J. Virol. 75: 6719-6728.
33 Ito, T., and Lai, M. M. C. 1999. Virology 254: 288-296.
34 Goodburn, S. et al. 2000. J. Gen. Virol. 81: 2341-2364.
35 Clemens, M. J., and Elia, A. 1997. J. Interf. and Cytok. Res.17; 503-524.
36 Behrens, S. E. et al. 1998. J. Virol. 72: 2364-2372.
37 Tautz, N. et al. 1999. J. Virol. 73: 9422-9432.
38 Lohmann, V. et al. 1999. Science 285:110-113.
39 Grassmann, C. W. et al. 1999. J. Virol. 73: 9196-9205.
40 Behrens, S. E. et al. 1996. EMBO J.15: 12-22.
41 Grassmann, C. W. et al. 2001. J. Virol.75: 7791-7802.
42 Yu, H., et al. 1999. J. Virol. 73: 3638-3648.
43 Yu, H., et al. 2000. J. Virol. 74: 5825-5835
44 Grassmann, C. W. et al. unpublished data.
45 Yu, H. et al. unpublished data.
46 Lu, H. H., and Wimmer, E. 1996. Proc. Natl. Acad. Sci USA 93: 1412-1417.
47 Deng, R., and Brock, K. V. 1993. Nucleic Acids Res. 21: 1949-1957.
48 Kolykhalov, A. A. et al., 1996. J. Virol. 70: 3363-3371.
49 Yanagi, M. et al. 1999. Proc. Natl. Acad. Sci. USA 96. 2291-2295.
50 Kolykhalov, A. A. et al. 2000. J. Virol. 74: 2046-2051.
51 Frolov, I. et al. 1998. RNA 4: 1418-1435.
52 Friebe, P. et al. 2001. J. Virol. 75: 12047-12057.
53 Isken, O. et al. unpublished data.
54 Ito, T. et al., 1998. J. Virol. 72: 8789-8796.
55 Brown, E. A. et al. 1992. Nucl. Acids Res. 20: 5041-5045.
56 Rijnbrand, R. et al. 1997. J. Virol. 71: 451-457
57 Barabino, S., and Keller, W. 1999. Cell 99: 9-11.
58 Kusov, Y. et al. 1996. J. Virol. 70: 1890-1897.
59 Le, S. Y. et al. 1996. Virus Genes 12: 135-147.
60 Corthesy, B. and Kao, P. 1994. J. Biol. Chem. 269: 20682-20690.
61 Kao, P. et al., 1994. J. Biol. Chem. 269: 20691-20699.
62 Saunders, L. R. et al., 2001. J. Biol. Chem. 276: 32300-32312.
63 Fierro-Monti, I., and Mathews, M. B. 2000. TIBS 25: 241-246.
64 Duchange, N. et al., 2000. Gene 261: 345-353.
65 Xu, Y. H. and Grabowsky, G. A. 1999. Mol. Genet. Metab. 68: 441-454.
66 Satoh, M. et al. 1999. J. Biol. Chem. 274: 34598-346404.
67 Parker, L. M. et al. 2001. J. Biol. Chem. 276:32522-32530.
68 Reichman, T. W. et al. 2002. Mol. Cell. Biol. 22: 343-356.
Liao, H. J. et al. 1998. Proc. Natl. Acad. Sci. USA 95: 8514-8519.

1. A method for modulating viral RNA replication and translation, in a eukaryotic cell, of positive-strand viral RNA, comprising the step of contacting a viral RNA-binding protein (vRbp) with a compound that modulates an activity of said vRbp.

2. The method of claim 1, wherein said vRbp is selected from the group consisting of: vRbp130, vRbp120, vRbp110, vRbp84, vRbp64, and vRbp45.

3. The method of claim 1 wherein said activity of the vRbp is selected from the group consisting of:
a response to viral RNA,
a response to interferon induction,
a response to double-stranded RNA-dependent protein kinase (PKR), and
a response to vRbp.

4. The method of claim 3 wherein said response is formation of a viral:cellular ribonucleoprotein (RNP) complex.

5. The method of claim 4 wherein said RNP complex comprises a viral RNA:vRbp interaction.

6. The method of claim 5 wherein said viral RNA:vRbp interaction comprises binding of a vRbp to a viral RNA 3′ untranslated region (3′UTR).

7. The method of claim 4 wherein said viral RNA:vRbp interaction comprises binding of a vRbp to a viral RNA 5′ untranslated region (5′UTR).

8. The method of claim 5 wherein said 3′UTR is a UGA box consensus sequence.

9. The method of claim 3 wherein said response is viral circularization.

10. The method of claim 9 wherein said viral circularization comprises binding of vRbp to the viral 5′UTR and 3′UTR creating a physical and functional link between both ends of the RNA.

11. The method of claim 9 wherein said viral circularization comprises an interaction between viral 5′UTR, 3UTR RNA, vRbp, and cellular proteins involved in the interferon antiviral response.

12. The method of claim 3 wherein said response is increase in translational frameshifting that result in decreased viral replication.

13. The method of claim 3 wherein said response is formation of a vRbp:PKR interaction.

14. The method of claim 1 wherein said viral replication and translation comprises coordinated regulation of replication and translation of viral RNA.

15. The method of claim 1, wherein said eukaryotic cell is a mammalian cell.

16-17. (canceled)

18. The method of claim 1, wherein said positive strand viral RNA comprises RNA from a member of the family Flaviviridae.

19. The method of claim 1 wherein said positive strand viral RNA comprises RNA from a member of the family Picornaviridae.

20-40. (canceled)

41. A method for modulating the function of a viral 3′UTR comprising the step of contacting a 3′UTR with a compound that modulates the structure of the 3′UTR as to inhibit the interaction between 3′UTR and vRbp.

42. A method for screening to identify compounds that activate or that inhibit the function of vRbp which comprises a method selected from the group consisting of:
(a) mixing a candidate compound with a solution containing a vRbp, to form a mixture, measuring activity of the vRbp in the mixture, and comparing the activity of the mixture to a standard;
(b) detecting the effect of a candidate compound on the production of viral RNA in a eukaryotic cell, using for instance, an ELISA assay, reticulocyte lysate translation assay (luciferase RNA); and
(c) (1) contacting a composition comprising the vRbp with the compound to be screened under conditions to permit interaction between the compound and the vRbp to assess the interaction of a compound, such interaction being associated with a second component capable of providing a detectable signal in response to the interaction of the vRbp with the compound; and
(2) determining whether the compound interacts with and activates or inhibits an activity of the vRbp by detecting the presence or absence of a signal generated from the interaction of the compound with the vRbp.

43-46. (canceled)
